(19) United States
(12) Patent Application Publication
(10) Pub. No.: US 2026/0337680 A1
(43) Pub. Date: Oct. 29, 2026

(54) Methods for Detection of Fetal Alcohol Spectrum Disorders

(71) Applicant: Rutgers, The State University of New Jersey, New Brunswick, NJ (US)

(72) Inventor: Dipak K. Sarkar, New Brunswick, NJ (US)

(73) Assignee: Rutgers, The State University of New Jersey, New Brunswick, NJ (US)

(21) Appl. No.: 19/545,894

(22) Filed: Feb. 20, 2026

Related U.S. Application Data
(60) Provisional application No. 63/761,643, filed on Feb. 21, 2025.

(51) Int. Cl. C12Q 1/6876

(52) U.S. Cl. CPC C12Q 1/6876

(57) ABSTRACT

Methods of detecting fetal alcohol spectrum disorders in a subject by measuring the expression patterns of one or more of circadian clock genes and their regulatory modifiers and kits are provided.

### CROSS-REFERENCE TO RELATED APPLICATION

The present application claims priority to U.S. Patent Application Ser. No. 63/761,643, filed Feb. 21, 2025, which is hereby incorporated by reference in its entirety.

### TECHNICAL FIELD

The present disclosure generally relates to methods of detecting fetal alcohol spectrum disorders in a subject by measuring the expression patterns of one or more circadian clock genes and their regulatory modifiers.

### BACKGROUND

Fetal alcohol spectrum disorders (FASD) include a wide range of physical, behavioral, and cognitive defects that occur due to alcohol exposure before birth. Some examples of FASD include fetal alcohol syndrome (FAS), partial FAS, alcohol-related neurodevelopment disorder (ARND) and neurobehavioral disorder¹. The prevalence of FASD is conservatively estimated to affect 11.3 to 50.0 per 1000 children in the United States, although the prevalence may be as high as 98.5 per 1000 children^{2,3}, and consequences include maladaptive behavioral characteristics, cognitive impairments, and physical abnormalities.⁴ In addition, children with FASD often show sleep problems,⁵ though limited work has been done to characterize these sleep disturbances and their consequences.⁶ Given the impact of sleep disturbance on neurobehavioral functioning³ and health,⁷ this is a significant, clinically relevant issue that is not well understood in the FASD population.

It is well recognized that the timing and duration of sleep are controlled by the circadian system, which keeps a roughly 24-hour internal rhythm that entrains to environmental stimuli, and the sleep homeostat.

Longitudinal studies suggest that individuals with FASD are at an increased risk for adverse long-term outcomes, including mental health problems and poor social adjustment.⁸ Despite extensive evidence of significant cognitive, emotional behavioral, and social deficits in people with FASD, therapeutic interventions for FASD are not developed. It is easy to misdiagnose FASD and research shows that diagnoses of autism and attention deficit hyperactivity disorders (ADHD) often precede an additional diagnosis of FASD.⁹ Diagnosing FASD is important as therapy and management for each of these disorders may differ, and expectations for outcomes is important.

Caregivers of children with FASD often report that their child has sleep problems, and many symptoms of sleep deprivation overlap with the cognitive and behavioral deficits characteristic of FASD. Given the impact of circadian disruption and sleep disturbance on neurobehavioral functioning and health, this is a significant clinically relevant issue that is not well understood in the FASD population. Prenatal alcohol-exposed (AE) infants and children often demonstrate disrupted sleep patterns, including more frequent awakenings, reduced total sleep time, and more night-to-night sleep variability. Despite the strong connection between sleep patterns and circadian rhythmicity, relatively little is known about circadian rhythm disruptions in individuals with AE.

Accordingly, there is a need to determine if an individual with sleep problems has FASD, and in particular a child, so that appropriate therapy and management of symptoms are applied.

### BRIEF DESCRIPTION OF THE DRAWINGS

FIG. 1 shows a diagram describing the mechanisms of modern daily circadian oscillation and identifying key activators and repressors.

FIG. 2 shows a flowchart describing protocols for performing expression analyses of human genes in saliva samples, including protocols for saliva sample collection, RNA isolation, conventional cDNA synthesis and high-capacity cDNA reverse transcription, adjustment of human RNA input for cDNA analysis, modified cDNA synthesis, and RTPCR in accordance with one or more embodiments.

FIGS. 3A-3X shows diagrams describing the effects of prenatal alcohol exposure on circadian expression of core clock genes (FIG. 3A-3U) and a peripheral circadian marker (FIG. 3V-3X). FIG. 3A, FIG. 3D, FIG. 3G, FIG. 3J, FIG. 3M, FIG. 3P, FIG. 3S, and FIG. 3V show changes in the rhythmic expression of the core clock genes BMAL1, CLOCK, PER1, PER2, PER3, CRY1, and CRY2, and the peripheral circadian marker ARR1B, respectively, in salivary samples collected at two time points (morning and afternoon) from each AE and control subjects throughout the day (ZT3 to ZT11) and plotted as spline curves. The diagrams in FIG. 3B, FIG. 3E, FIG. 3H, FIG. 3K, FIG. 3N, FIG. 3Q, FIG. 3T, and FIG. 3W show changes in the expression of the core clock genes BMAL1, CLOCK, PER1, PER2, PER3, CRY1, and CRY2, and the peripheral circadian marker ARR1B, respectively, when plotted as mean±SEM at each hour during ZT 3:00 to ZT 11:00 in AE. Data were analyzed using Two-way ANOVA and the interaction between the treatment and time were identified. FIG. 3C, FIG. 3F, FIG. 3I, FIG. 3L, FIG. 3O, FIG. 3R, FIG. 3U, and FIG. 3X show the overall Mean±SEM levels of gene expression in two groups for the core clock genes BMAL1, CLOCK, PER1, PER2, PER3, CRY1, and CRY2, and the peripheral circadian marker ARR1B, respectively, were analyzed using one-way ANOVA. *** represents a p-value of <0.001.

FIGS. 4A-4R shows diagrams describing the effects of prenatal alcohol exposure on circadian expression of an activator NPAS2 (FIG. 4A-FIG. 4C), and repressors (NFL3, FIG. 4D-FIG. 4F; NR1D1, FIG. 4G-FIG. 4I; DEC1, FIG. 4J-FIG. 4L; DEC2, FIG. 4M-FIG. 4O; DBP, FIG. 4P-FIG. 4R) of transcription/translation of core circadian control genes via its role in a negative feedback loop responsible for the control of circadian rhythm. FIG. 4A, FIG. 4D, FIG. 4G, FIG. 4J, FIG. 4M, and FIG. 4P show changes in the rhythmic expression of the activator NPAS2 and the core circadian control genes NFL3, NR1D1, DEC1, DEC2, and DBP, respectively, from each AE and control subjects throughout the day and plotted at the time of collections during ZT 3:00 to ZT 11:00 using spline curves. FIG. 4B, FIG. 4E, FIG. 4H, FIG. 4K, FIG. 4N, and FIG. 4Q changes in the expression of activator NPAS2 and the core circadian control genes NFL3, NR1D1, DEC1, DEC2, and DBP, respectively, when plotted as mean±SEM at each hour during ZT 3:00 to ZT 11:00. The n values are the same as those discussed in FIG. 3.

FIGS. 5A-5R shows diagrams describing the effects of prenatal alcohol exposure on circadian expression of various epigenetic modulators known to interact with CLOCK:BMAL1 and impose oscillatory patterns to the epigenetic mark at the promoter of PERICRY gene to maintain circadian rhythmicity, such as the transcription activators MLL1 (FIG. 5A-FIG. 5C) and P300 (FIG. 5D-FIG. 5F); and the transcription inhibitors SIRT1 (FIG. 5G-FIG. 5I), EZH2 (FIG. 5J-FIG. 5L), HDAC3 (FIG. 5M-FIG. 5O), and JARID1 (FIG. 5P-FIG. 5R). FIG. 5A, FIG. 5D, FIG. 5G, FIG. 5J, FIG. 5M, FIG. 5P show changes in the rhythmic expression of the epigenetic modulators of core clock genes MLL1, P300, SIRT1, EZH2, HDAC3, and JARID1, respectively, in salivary samples collected at two time points (morning and afternoon) from each AE and control subjects throughout the day and plotted at the time of collections during ZT 3:00 to ZT 11:00. FIG. 5B, FIG. 5E, FIG. 5H, FIG. 5K, FIG. 5N, and FIG. 5Q show changes in the expression of the epigenetic modulators of core clock genes MLL1, P300, SIRT1, EZH2, HDAC3, and JARID1, respectively, when plotted as mean±SEM at each hour during ZT 3:00 to ZT 11:00. The n values are the same as those discussed in FIG. 3.

FIGS. 6A-6H show diagrams describing the changes in the Mean+SEM levels of core clock genes (FIG. 6A: BMAL; FIG. 6B: CLOCK; FIG. 6C: PER1; FIG. 6D: PER2; FIG. 6E: PER3; FIG. 6F: CRY1; FIG. 6G: CRY2) and peripheral circadian marker ARR1B (FIG. 6H) in male and female control and FASD (prenatal ethanol exposed) children.

FIGS. 7A-7F show diagrams describing the changes in the Mean+SEM levels of an activator NPAS2 (FIG. 7A), and repressors NFL3 (FIG. 7B), NR1D1 (FIG. 7C), DEC1 (FIG. 7D), DEC2 (FIG. 7E), and DBP (FIG. 7F) of transcription/translation of core circadian control genes in male and female control and FASD (prenatal ethanol exposed) children.

FIGS. 8A-8F shows diagrams describing the changes in the Mean+SEM levels of various epigenetic modulators known to interacts with CLOCK:BMAL1 and imposes oscillatory pattern to the epigenetic mark at the promoter of PER/CRY gene to maintain circadian rhythmicity in male and female control and FASD (prenatal ethanol exposed) children (n; Male—Control, 21; −FASD, 24; Female—Control, 30; FASD, 22), including the transcription activators MLL1 (FIG. 8A) and P300 (FIG. 8B), and the transcription inhibitors SRT1 (FIG. 8C), EZH2 (FIG. 8D), HDAC3 (FIG. 8E), and JARID1 (FIG. 8F).

FIG. 9 shows a comparison plot of AF/V (alcohol fed-vector treated), AFKD (AF-Dppa4 knockdown), and ADKI (control-fed-DPPA4 over expressed) with respect to AD/V (control fed-vector treated) and shows the effect of DPPA4 on the expression of different circadian genes. Data were obtained from RNA-seq of rat pituitary cells, with n=3.

FIGS. 10A-10H show comparison plots which demonstrate that the promoters of different circadian gene are regulated by DPPA4. ChIP assays data show the binding of DPPA4 in the promoter region of (FIG. 10A) BMAL, (FIG. 10B) CLOCK, (FIG. 10C) NPAS2, (FIG. 10D) PER1, (FIG. 10E) PER2, (FIG. 10F) PER3, (FIG. 10G) CRY1, and (FIG. 10H) CRY2. Data were analyzed using one-way or two-way ANOVA with the Newman Keuls post-hoc test or Dunnett's multiple comparison test. (n=6), **p<0.01 and ***p<0.001 vs AD as indicated by a line above the bar.

FIGS. 11A-11H show diagrams that demonstrate that expression levels of DPPA4 in saliva show a circadian rhythm pattern in control but not in FASD. FIG. 11A and FIG. 11B show the effects of AE on circadian expression of DPPA4 levels in saliva for control (FIG. 11A) and FASD (FIG. 11B) groups when plotted individually at various timepoint using a Sinusoidal curve. FIG. 11C and FIG. 11D show the results of a cosinor analysis used to determine the circadian rhythm in control (FIG. 11C) and FASD (FIG. 11D) groups. A Zero-amplitude test p-value of <0.05 identified rhythmicity and the Acrophase value shows the time when the rhythm peaked. FIGS. 11E-11H show gene expression levels of DPPA at various timepoints for control and FASD groups individually as Mean+SEM value at each time point (FIG. 11E-FIG. 11F), combined (FIG. 11G), or separated by sex (FIG. 11H).

FIGS. 12A-12X. Expression levels of a peripheral molecular marker of circadian rhythm (ARRB1) and core clock genes (BMAL1, CLOCK, PER1,2,3, CRY1,2) in saliva demonstrate circadian rhythm patterns in control (CON) children, but not in alcohol-exposed (PAE) children: Effects of PAE on circadian expression of peripheral circadian markers (FIGS. 12A-12C) and core clock genes (FIGS. 12D-12X). Cosinor analysis was used to determine the circadian rhythm of these clock genes (Zero-amplitude test p value <0.05 identified rhythmicity and the Acrophase value shows the time when the rhythm peak). Mean+SEM value of gene expression levels are analyzed using one-way ANOVA *, p<0.05; **, p<0.01; ***, p<0.001. Y. Schematic diagram showing postulated mechanisms by which PAE alters circadian oscillation by modifying expression of core clock genes in children. The daily circadian oscillation which is maintained by a coordinated transcription-translation feedback loop between the core clock activators (BMAL and CLOCK) and core clock repressors (PER and CRY) is affected by PAE. Down arrow indicates inhibition.

FIGS. 13A-13H. Effects of PAE on circadian expression of peripheral circadian markers (FIG. 13A) and core clock genes (FIGS. 13B-13H) in male (n=PAE-24; Control-21) and female (n=PAE-20; Control-32) children. Mean+SEM value of gene expression levels are analyzed using one-way ANOVA *, p<0.05; **, p<0.01; ***, p<0.001. Very little sex difference in PAE effect on circadian genes are observed with exception of CLOCK gene.

FIGS. 14A-14P. Gene level variations throughout the day in control or FASD group did not change whether it is plotted individually (FIGS. 14A, 14C, 14E, 14G, 14I, 14K, 14M, 14O) or as a group (FIGS. 14B, 14D, 14F, 14H, 14J, 14L, 14N, 14P). n=3-10.

### SUMMARY

In a first aspect, a method for detection of fetal alcohol spectrum disorders in a subject is provided. In the method, at least one saliva sample from the subject is combined with a DNA/RNA shield solution to form a mixture and the mixture is centrifuged. Ethanol is added to the mixture and the mixture is further centrifuged. The mixture is then washed with a DNA/RNA buffer solution. A DNase/RNase-free water solution is mixed into the mixture. RNA is then collected from the mixture. The collected RNA is then converted into cDNA via reverse transcription. A quantitative reverse transcription polymerase chain reaction (qRT-PCR) is then performed on the cDNA to measure gene expression patterns of one or more genes. Dysregulation of the expression patterns of the one or more genes is indicative of fetal alcohol spectrum disorder in the subject.

In another aspect, the subject is a human. In another aspect, the subject is approximately 6-10 years old.

In another aspect, the at least one saliva sample and the DNA/RNA shield solution are mixed in approximately a 1:1 ratio.

In another aspect, the mixture is first centrifuged for approximately 2 minutes. In another aspect, the washing of the mixture comprises two washings with the DNA/RNA buffer solution.

In another aspect, the collected RNA is converted into cDNA via reverse transcription using a high-capacity cDNA reverse transcription kit.

In another aspect, the one or more genes comprise clock genes.

In another aspect, the one or more genes are selected from the group consisting of BMAL1, CLOCK, ARRB1, PER1, PER2, PER3, CRY1, CRY2, NPAS2, NFL3, NR1D1, DEC1, DEC2, DBP.

In another aspect, the one or more genes include BMAL1 and wherein relative expression of BMAL1 above a normal range is indicative of fetal alcohol spectrum disorder in the subject.

In another aspect, the one or more genes include CLOCK and wherein relative expression of CLOCK above a normal range is indicative of fetal alcohol spectrum disorder in the subject.

In another aspect, the one or more genes include at least one of PER1, PER2, CRY1, CRY2, and ARRB1, and wherein relative expression of at least one of PER1, PER2, CRY1, CRY2, and ARRB1 below a normal range is indicative of fetal alcohol spectrum disorder in the subject.

In another aspect, expression patterns of one or more chromatin modifiers are measured via the qRT-PCR performed on the cDNA, wherein the chromatin modifiers are selected from the group consisting of MLL1, P300, SIRT1, EZH2, HDAC3, and JARID1.

In a second aspect, a kit for detection of fetal alcohol spectrum disorders in a subject via a saliva sample is provided. The kit comprises a saliva collection device, a DNA/RNA buffer solution, a DNA/RNA shield solution, a DNase/RNase-free water solution, and a cDNA reverse transcription kit. The cDNA reverse transcription kit is configured to convert collected RNA into cDNA.

In another aspect, the cDNA reverse transcription kit comprises a buffer, a deoxynucleotide triphosphate mix, random primers, and reverse transcriptase.

In another aspect, the cDNA reverse transcription kit is a high-capacity cDNA reverse transcription kit.

In another aspect, the saliva collection device comprises a collection funnel and a collection tube.

### DETAILED DESCRIPTION OF CERTAIN EMBODIMENTS

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The term “prenatal alcohol exposure” (PAE or AE, which are used interchangeably herein) generally refers to exposure to alcohol during gestation via maternal alcohol use during pregnancy. Heavy prenatal alcohol exposure is further defined as PAE or AE that results in alcohol-attributable diagnoses given to the mother or her child, or by maternal redeemed prescriptions for drugs to treat alcohol dependence 1 year before or during pregnancy.

“Clock genes” are generally defined as genes that serve as the basis of the intracellular timekeeping system, which promotes time-based oscillations in the transcription of certain genes across cells.

The term “about” or “approximately” means within an acceptable error range for the particular value as determined by one of ordinary skill in the art, which depends in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

In accordance with one or more embodiments, the present application provides methods for detecting fetal alcohol spectrum disorder in a subject. In one or more embodiments, the subject is a human. In one or more embodiments, the humans are children or adolescents. In one or more embodiments, the children or adolescents are aged 1-15 years, 1-10 years, or 6-10 years.

In accordance with one or more embodiments, in a first aspect of the method, at least one saliva sample is collected from the subject. In one or more embodiments, the saliva sample(s) can be collected from the subject in numerous ways, including as further described in the examples below, such as via saliva/sputum collection kits. Once the saliva sample is collected from the subject, the saliva sample is then combined with a DNA/RNA shield solution to form a mixture. The DNA/RNA shield solution is a reagent for nucleic acids in any biological sample that helps to preserve the genetic integrity of the saliva samples, as is known and understood in the art. In one or more embodiments, the saliva sample and the DNA/RNA shield solution are mixed in approximately a 1:1 ratio.

In one or more embodiments, the mixture of the saliva sample and the DNA/RNA shield solution are then centrifuged. In one or more embodiments, the mixture is centrifuged for approximately 1-5 minutes. In at least embodiment, the mixture is centrifuged for approximately 2 minutes.

In at least one embodiment, ethanol can be subsequent added to the mixture, and the mixture can undergo further centrifuging.

In one or more embodiments, the mixture can then be washed with a DNA/RNA buffer solution. In at least one embodiment, the washing of the mixture with the DNA/RNA buffer solution can be repeated such that there are two washings of the mixture.

In one or more embodiments, after washing, the mixture is further mixed with a DNase/RNase-free water solution.

In one or more embodiments, RNA is then collected from the mixture, and then converted into cDNA via reverse transcription. In at least one embodiment, the collected RNA is converted into cDNA via reverse transcription using a high-capacity cDNA reverse transcription kit.

After conversion of the RNA into cDNA, a quantitative reverse transcription polymerase chain reaction (qRT-PCR) can be performed on the cDNA to measure gene expression patterns of one or more genes. In one or more embodiments, the genes one include clock genes. In one or more embodiments, the one or more genes can include BMAL1, CLOCK, ARRB1, PER1, PER2, PER3, CRY1, CRY2, NPAS2, NFL3, NR1D1, DEC1, DEC2, and DBP.

In one or more embodiments, the expression patterns of one or more chromatin modifiers can also be measured. The expression patterns of the chromatin modifiers can be measured in the same or similar fashion as the expression patterns of the genes (e.g., clock genes) are measured, such as via qRT-PCR performed on the cDNA. In one or more embodiments, the chromatin modifiers can include MLL1, P300, SIRT1, EZH2, HDAC3, and JARID1.

Once the gene expression patterns in the genes (e.g., clock genes) and/or the chromatin modifiers have been measured, it is then determined whether the expression patterns of those genes and/or chromatin modifiers have been disrupted or dysregulated. Disruption or dysregulation of the expression patterns of the one or more genes and/or chromatin modifiers can be indicative of fetal alcohol spectrum disorder in the subject.

In one or more embodiments, disruption or dysregulation of the expression patterns of the genes and/or chromatin modifiers is measured over time to determine whether the level of disruption or dysregulation in the gene or chromatin modifier is indicative of fetal alcohol spectrum disorder. For example, in accordance with one or more embodiments, the following Table 1 provides the relative expression levels of genes (e.g., clock genes) at various time points during the day that are indicative of FASD. Similarly, Table 2 indicates the typical ranges of relative expression of the various genes at the same time points and whether dysregulation or disruption of the particular genes associated with FASD results in lower or higher expression of the gene.

TABLE 1
Relative Expression of Genes at Hours 10:00, 13:00, and 16:00
Indicating FASD (relative expression values are approximate values).

| | Gene | | | | | | |
|---|---|---|---|---|---|---|---|
| Time | name | | | | | | |
| (hour) | Bmal1 | CLOCK | PER1 | PER2 | CRY1 | CRY2 | ARR1B |
| 10:00 | 2.0-2.5 | 2.0-3.0 | 0.5-1.0 | 1.0-1.5 | 1.0-1.5 | 0.5-1.0 | 0.5-1.0 |
| 13:00 | 2.0-2.5 | 2.0-3.0 | 0.5-1.0 | 1.0-1.5 | 1.0-1.5 | 0.5-1.0 | 0.5-1.0 |
| 16:00 | 1.5-2.0 | 1.5-2.0 | 0.5-1.0 | 1.0-1.5 | 1.0-1.5 | 0.5-1.0 | 0.5-1.0 |

TABLE 2
Typical Ranges of Relative Expression of Genes at Hours 10:00, 13:00, and
16:00 Compared with Dysregulated or Disrupted Expression Associated with FASD
(typical values/ranges of relative expression are approximate values/ranges).

| | Gene name | | | | | | |
|---|---|---|---|---|---|---|---|
| | (normal | | | | | | |
| | expression | | | | | | |
| Time | range) | | | | | | |
| (hour) | Bmal1 | CLOCK | PER1 | PER2 | CRY1 | CRY2 | ARR1B |
| 10:00 | Higher > | Higher > | Lower < (2.0) | Lower < (2.0) | Lower < (2.0) | Lower < (2.0) | Lower < (2.0) |
| | (1.0-1.5) | (1.0-1.5) | | | | | |
| 13:00 | Higher > | Higher > | Lower < (2.0) | Lower < (2.0) | Lower < (2.0) | Lower < (2.0) | Lower < (2.0) |
| | (0.5-1.0) | (1.0-1.5) | | | | | |
| 16:00 | Higher > | Higher > | Lower < (2.0) | Lower < (2.0) | Lower < (2.0) | Lower < (2.0) | Lower < (2.0) |
| | (1.0-1.5) | (1.0-1.5) | | | | | |

As exemplified in Tables 1 and 2, at all 3 time points, higher relative expression of Bmal1 and CLOCK genes was indicative of FASD. In contrast, at all 3 time points, lower relative expression of PER1, PER2, CRY1, CRY2, and ARR1B genes was indicative of FASD.

Thus, in one or more embodiments of the present methods, a finding of relative expression of BMAL1 and/or CLOCK genes that is above a normal range (see Table 2) and/or in the ranges shown in Table 1 at the various time points is indicative of fetal alcohol spectrum disorder in the subject. Likewise, in one or more embodiments, a finding of relative expression of at least one of PER1, PER2, CRY1, CRY2, and ARRB1 that is below a normal range (see Table 2) and/or in the ranges shown in Table 1 at the various time points is indicative of fetal alcohol spectrum disorder in the subject.

In one or more embodiments, if FASD is detected in the subject, the subject can then be treated. While there is no known cure for FASD, subjects with FASD can be treated with one or more of cognitive therapies, behavioral therapies, and sleep-based therapies, for example. Early diagnosis of FASD can improves outcomes for patients, particularly children. Accordingly, the present method, which can provide an early detection of FASD in children is a crucial first step in the treatment process for patients with these conditions.

In one or more embodiments, a kit for detection of fetal alcohol spectrum disorders in a subject via a saliva sample is provided. The kit can include a saliva collection device, a DNA/RNA buffer solution, a DNA/RNA shield solution, a DNase/RNase-free water solution, and a cDNA reverse transcription kit configured to convert collected RNA into cDNA. In at least one embodiment, the cDNA reverse transcription kit can include a buffer, a deoxynucleotide triphosphate mix, random primers, and reverse transcriptase. In at least one embodiment, the cDNA reverse transcription kit is a high-capacity cDNA reverse transcription kit. In one or more embodiments, the saliva collection device comprises a collection funnel and a collection tube.

These and other features of the present methods and kits for detecting fetal alcohol spectrum disorder are now described in further detail, with reference to the accompanying figures and examples.

### Daily Circadian Oscillation and Clock Genes

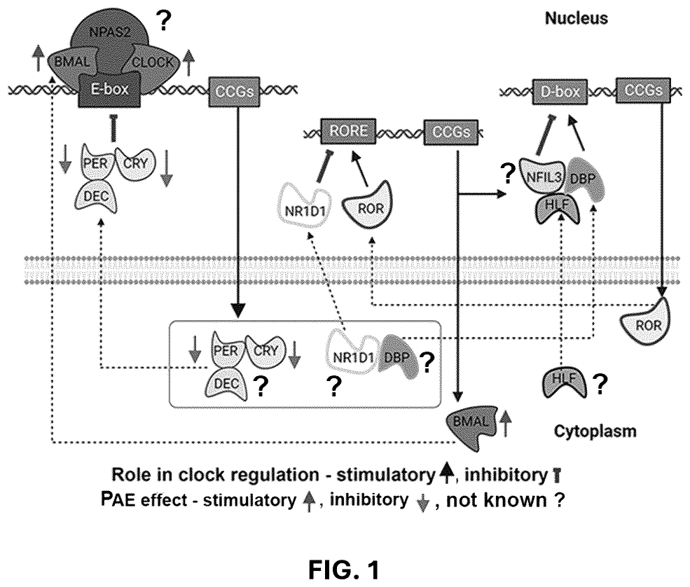

FIG. 1 shows a diagram describing the mechanisms for modern daily circadian oscillation and identifying key activators and repressors. Daily circadian oscillation is maintained by a coordinated transcription-translation feedback loop between the core clock activators (BMAL and CLOCK, or BMAL and NAPS2) and core clock repressors (PER and CRY). The other negative regulators of the molecular clockwork, DEC proteins, are also shown to inhibit CLOCK-BMAL1-mediated transcription of E-box-containing promoters. Also, NR1D and RORE proteins which compete to bind RORE are known to alter the expression of various circadian clock genes (CCGs), including BMAL. Additional regulators controlling this circuitry include activator DBP and HLF and the repressor NFIL3 through the D-box element. Postulated sites of AE action on the clock regulatory genes are shown by question marks.

The circadian system is a dynamic biological timekeeper system that controls daily timings of physiological, biochemical, and behavioral functions.¹⁰ At the molecular level, it consists of various clock genes that participate in maintaining a 24-hour internal timekeeping. The core clock genes BMAL1 (Brain and muscle arnt-like1) and CLOCK (from circadian locomotor output cycles kaput) rhythmically regulate expression of other core clock genes that encode PER (period) 1/2/3 and CRY (Cryptochrome Circadian Regulator) 1/2 proteins. The PER/CRY complex subsequently translocates to the nucleus from cytoplasm to suppress transcription of BMAL1/CLOCK, thus completing a transcription-translation negative feedback loop that occurs over a 24-hour period. Expression of these clock genes within the hypothalamic suprachiasmatic nucleus (SCN), the mammalian brain's circadian central pacemaker, and other peripheral pacemaker cells, integrate SCN temporal information into physiological processes. The molecular composition of the timing mechanisms in the SCN clock and peripheral oscillators is highly resembling, despite region-specific differences in particular gene products to achieve local physiological programs.¹¹ Therefore, clock genes serve as the basis of an intracellular timekeeping system, present throughout the body, which generates approximately 24-hour rhythms in physiology and behavior. Many clock-controlled genes show circadian oscillation in their mRNA expression patterns, resulting in numerous physiological processes that exhibit circadian rhythmicity.¹²

In addition to core clock genes, several additional genes and feedback loops participate in fine tuning the rhythmic expression of clock genes and clock-regulated physiological functions. For example, BMAL1 expression is regulated by the secondary feedback loop, in which CLOCK and BMAL1 induce NR1D1 (Nuclear Receptor Subfamily 1 Group D Member 1 also known as REV-ERBα) and NR1D2 (REV-ERBβ) expression and then NR1D1/NR1D2 proteins repress BMAL1 expression 13-15. The other negative regulators of the molecular clockwork, DEC1 and DEC2 (also known as Basic helix-loop-helix family member e41, or BHLHE41) are also shown to inhibit CLOCK-BMAL1-mediated transcription of E-box-containing promoters.¹⁶ Additional motifs involved in rhythmic gene expression are D-box elements, which are activating transcription factors DBP (D-box binding protein), and the repressor NFIL3 (nuclear factor, interleukin-3 regulated). DBP rhythmically activates transcription of various genes through a DNA cis-element, D-box. The DBP-dependent transactivation is repressed by competitive binding of NFIL3 to the D-box. Collectively, D-box-mediated transcriptional regulation plays important roles in input and output in the circadian clock system.^{17,18} Finally, neuronal PAS domain protein 2 (NPAS2) has been shown to bind with DNA at E-box sequences and form heterodimers with BMAL1 to control cellular rhythmicity.¹⁹

Available data indicate the relevance of circadian dysregulation associated with drug addiction, including alcohol.²⁰⁻²³ In a study employing male patients with alcohol dependence, it has been shown that basal levels of circadian genes are significantly reduced in peripheral blood mononuclear cells (PBMC) of alcohol-dependent patients.²⁴ However, the effect of prenatal ethanol on circadian clock gene expression has been mainly studied in experimental animals.²⁵⁻²⁸ There are a number of studies indicating that prenatal alcohol exposure affects circadian expression of hormones, body temperature, blood pressure, and regulation of the stress response,^{25,29,30} including a study that used a rat model of prenatal alcohol exposure to show altered circadian expression of clock genes, Per1 and Per2, in the arcuate nucleus and the SON of male rats.^{20,26} However, this has not been investigated in clinical populations.

Human saliva is an excellent source of genetic (DNA/RNA), protein, and metabolite which makes it an ideal biofluid for non-invasive clinical assessment of systemic diseases.³⁴ Saliva sampling is also less stressful and is thus ideal for sequential sampling. In fact, salivary cortisol level was measured in children with FASD and found to be higher across the day compared to children without FASD.³⁵ ARRB1 (β-arrestin1) has been identified to function as a circadian salivary biomarker which plays an important role in circadian regulation of diverse physiological functions.¹³ However, there are no studies available wherein FASD saliva transcripts were analyzed in connection to circadian rhythmic pattern.

In the present application, it is reported for the first time that core-clock genes are expressed in a diurnal pattern in saliva, and that prenatal ethanol exposure significantly alters the circadian expression of these clock genes. Additionally, in the present application, the role of chromatin modifiers in the circadian expression of clock genes has been identified, uncovering likely molecular mechanisms triggering pathological states associated to circadian misalignment by prenatal exposure to ethanol.

In several examples of the present application, and in accordance with one or more embodiments of the present methods, the expression patterns of core-clock genes and their regulatory genes in salivary samples of children aged 6-10 years-old with and without AE are compared during the light cycle between ZT0-ZT11. In these examples, the RNA from the samples were isolated and the expression patterns of core clock genes and clock regulating genes were measured using the human specific primers with quantitative real-time PCR. The gene expression profile of a salivary circadian biomarker ARRB1 was rhythmic in saliva of control children but was arhythmic in AE children.

Analysis of core clock genes expression levels in saliva samples from AE children indicates significantly altered levels in expression of core-clock BMAL1, CLOCK, PER1-3 and CRY1,2, as compared to those in age-matched control children. No sex difference in levels of clock genes were found in AE and control groups. Cosinor analysis was used to evaluate the rhythmic pattern of these clock genes, which identified circadian patterns in the levels of core clock genes in the control group but absent in the AE group. Altered expression patterns were also observed in clock regulatory genes: NPAS2, NFL3, NR1D1, DEC1, DEC2, and DBP, as well as chromatin modifiers: MLL1, P300, SIRT1, EZH2, HDAC3, and JARID1, known to maintain rhythmic expression of core-clock genes. Overall, these findings provide the first evidence that AE disturbs the circadian patten expression of core clock genes and clock-regulatory chromatin modifiers in saliva.

In addition, other examples of the present application employ an established preclinical model of FASD in which pregnant rats are fed alcohol or control liquid diets to induce many FASD-related behaviors in offspring. The aims of these examples are to: 1) confirm the finding that children with FASD have altered circadian expression of core clock genes and their regulatory factors in saliva samples and relate to behavioral outcome; 2) determine the transcriptomic changes in saliva of children with FASD for understanding the molecular mechanism involved in circadian system impairment and also to identify if genes associated with stress, anxiety and depression are differentially expressed in FASD and control children; and 3) identify if a newly discovered AE responsive gene, DPPA4, is involved in AE-induced alteration in circadian rhythmicity and behavioral abnormalities in a preclinical animal model of FASD.

The methods of the present application are highly innovative as saliva samples with protected RNAs and DNAs are a noninvasive method for clinical assessment of the acyclicity of circadian clock genes. No prior research has tested whether salivary samples of alcohol-exposed children show circadian clock disruption, nor evaluated the changes in transcriptome related to behavioral abnormalities of FASD for an early diagnosis.

Additionally, the methods of the present application indicate that saliva transcripts change along with the circadian rhythmic pattern and utilize the core-clock gene's diurnal pattern in saliva, which changes significantly under the influence of prenatal ethanol exposure. The present methods also identify the role of chromatin modifiers in the circadian expression of clock genes, and a strong association between disruption of circadian rhythms and psychiatric illness, anxiety, and hyper-reactive stress responses in children with FASD. Accordingly, the methods of the present application describe the detection of circadian abnormalities and restoration of circadian rhythm in preclinical FASD for early intervention to attain the normal development of an FASD child.

The following examples of the present application indicate that human saliva offers an emerging and easily available physiological sample that can be collected non-invasively for core-clock gene transcript analyses.

### EXAMPLES

### Example 1

### Experimental Details—Evaluating the Effect of AE in Circadian Clock Protein Expression in Children

Human Subjects. Children aged 6-10 years, with and without heavy prenatal alcohol exposure (alcohol-exposed [AE], control [CON]), were recruited from the FASD Research Registry, the San Diego State University Center for Behavioral Teratology and the University of California San Diego (UCSD) FASD Research Registry, and the MotherToBaby program. Subjects were enrolled as part of a larger study examining sleep and neurobehavioral functioning in individuals with heavy prenatal alcohol exposure. Two groups of children are recruited and assessed: children with histories of heavy prenatal alcohol exposure (AE; n=23) and typically developing children (CONT; n=26). Children of all sexes, races, and ethnicities between 6:0-10:11 years of age was selected based on the inclusion and exclusion criteria described in previous studies that are hereby incorporated by reference.^{7,36}

Two saliva KITs (Zymo Research saliva/sputum collection kit; R1210) were sent to each participant. Parents were instructed to collect saliva samples at 2 time points during the daytime (ZT3-11) for each child. One child in the control group gave only one saliva sample. All methods were carried out in accordance with relevant guidelines and regulations. All experimental protocols underwent Institutional Review Board (IRB) approval from University of California San Diego/San Diego State University. Informed consents were obtained from all subjects and/or their legal guardians.

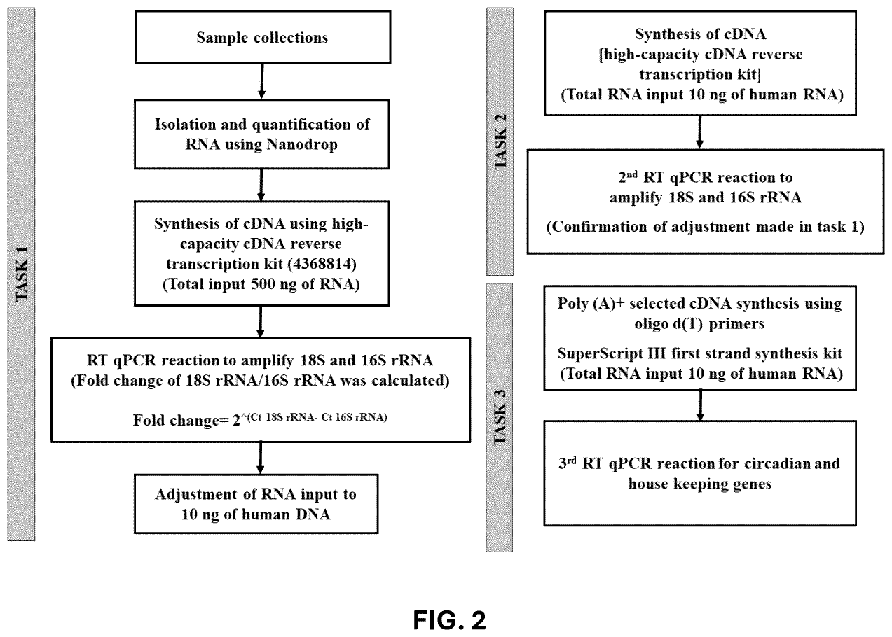

FIG. 2 describes a flowchart describing protocols for saliva sample collection, RNA isolation, conventional cDNA synthesis and high-capacity cDNA reverse transcription, adjustment of human RNA input for cDNA analysis, modified cDNA synthesis, and RTPCR in accordance with one or more embodiments herein. These protocols are discussed in detail below.

Saliva sample collection. The saliva samples from control and AE children were collected at different time points. Saliva/sputum collection kits (R1210) were used to collect saliva samples in specified containers containing DNA/RNA shield solution which protected the DNA/RNA present in the samples. The samples and shield solutions were mixed at a ratio of 1:1 according to the manufacturer's instructions. A total 51 control and 46 AE samples were collected. Time of collection was recorded, and kits with saliva samples were collected.

RNA isolation. The RNA isolation was done according to the protocol of Zymo Research kit (D7005). In brief, to the 1:1 mixture of saliva and shield solution Proteinase K and Proteinase K Digestion Buffer were added. The whole mixture was then incubated at room temperature (20-30° C.) for 30 minutes. After incubation, samples were vortexed and centrifuged at maximum speed for 2 minutes to pellet debris. The 400 ml of the cleared supernatant was transferred to the nuclease-free tube. The DNA/RNA lysis buffer was added at the ratio of 1:1 and mixed well. The samples were then transferred into the Zymo-Spin™M IC-XM Column1 with the collection tube and centrifuged to flow-through which contained the RNA. The flow-through was then mixed with one volume of 95% to 100% ethanol. After mixing, the samples were transferred into the Zymo-Spin™ IC Column′ with the collection tube and centrifuged. The flow-through was discarded and the column was washed with 400 μl of DNA/RNA prep buffer followed by 2 washings with DNA/RNA wash buffer. After washing, 15 μl of DNase/RNase-free water was added directly to the column matrix and centrifuged to collect the RNA and used immediately for cDNA preparation or stored frozen.

Conventional cDNA synthesis and high-capacity cDNA reverse transcription. The following strategy was taken according to a previously published protocol that is hereby incorporated by reference.³⁷ For analyzing the expression of pan-bacterial rRNA (16S), and human rRNA (18S), total salivary RNA was converted into complementary DNA (cDNA) via reverse transcription using the High-capacity cDNA reverse transcription kit (Applied Biosystem, Life Technologies). The RNA concentration was measured in NanoDrop and total 500 ng of RNA was used as input for all the samples. After reverse transcription, cDNA was diluted and used for qRT-PCR for the detection of 16S and 18S rRNAs.

Adjustment of human RNA input for cDNA synthesis. The adjustment of input was done according to a previously published protocol that is hereby incorporated by reference.³⁷ As NanoDrop spectrophotometer only provides total RNA values which consists of the mixture of human and bacterial RNA, the ratio of the detected raw Ct-values of human rRNA (18S) to pan-bacterial rRNA (16S) was calculated. The rRNA is representative for all human and bacterial RNA specimens. The ratio was used to calculate fold change value using the following formula: Fold change=2\hat{ }(Ct18S rRNA-Ct16S rRNA). After that, the total amount of RNA loaded for each sample (500 ng) was divided with fold change value which gave the amount of human RNA in individual sample. Finally, normalization was done based on the amount of human RNA (5 ng) and samples were used to synthesized cDNA using oligo d (T) primers.

Modified cDNA synthesis—SuperScript III First-Strand Synthesis System. To perform gene expression analysis of human origin only, the eukaryotic messenger RNA (mRNA) was reverse transcribed to cDNA by using the SuperScript® III First-Strand Synthesis System with Oligo (dT) primers.³⁷ As a result, pan-bacterial RNA and other human RNA species like tRNA, rRNA were excluded from cDNA conversion. According to the kit description, the amount of starting material can vary from 1 μg to 5 μg of RNA. The amount of 5 ng of human RNA per reaction was set to synthesize cDNA. Using the concentration from repeated NanoDrop™ measurements and the calculated 18S/16S ratio in each sample, the corresponding amount of measured total RNA input was calculated. The reverse transcription was performed according to the manufacturer's instructions.

RT PCR (qPCR). The c-DNA was prepared from the RNA of each sample and 1:3 dilution was done using nuclease free water. The real-time qPCR was carried out in 384 well plates. Each well of 384 well plates contain a reaction mixture of 10 μl consisting of 1:3 dilution of c-DNA, primers in Power SYBR Green PCR Master Mix and comparative CT (ΔΔCT) standard run method was carried out in Quant Studio 7 real time PCR system. The expression of genes was expressed in terms of relative quantification using the formula 2\hat{ }−(ΔΔCT).³⁸ The sequences of the primers used in this procedure are annotated in Table 3, below.

TABLE 3
Primer sequences for qPCR analysis
of salivary clock gene

| Human Primers | | |
|---|---|---|
| for qPCR | Sequences (5′ to 3′) | |
| PER1 | F | AGCACATCACGTCTGAGTACA |
| | R | TGCCTGCTCCGAAATGTAGA |
| PER2 | F | GAACTGCCCCTGGACTAAGAAA |
| | R | TTTGACCCGCTTGGACTTCA |
| PER3 | F | TGGTGGTGGTGAATGTAAGACC |
| | R | TGCTCATCGTTCCTCAAATCCT |
| BMAL | F | CCAGTAATGATGAGGCAGCAATG |
| | R | GCAAGTCACTAAAGTCAACAGGG |
| CLOCK | F | CACAGTCAGTCTCAAGGAAGCA |
| | R | ACGGCCGTGTGAGATGATTT |
| CRY1 | F | ACTATGCTCATGGCGACAGTCA |
| | R | ACTAGGACGTTTCCCACCACTG |
| CRY2 | F | GTGCACTGGTTCCGCAAAGG |
| | R | CACGGGTCGAGAATGTAAACGC |
| DBP | F | TCTTGCCCTATCAAGCATTCCT |
| | R | TTCATGATTGGCTGGGGCTT |
| DEC1 | F | AAGACGGGGAATAAAGCGGA |
| | R | TGCACTCGTTAATCCGGTCA |
| DEC2 | F | CAACTCTGGGACATCTGGAGAAA |
| | R | TATCTTCTGATGCTGTTGCTCGG |
| HLF | F | CGCAAACGCAAGTTCTCTGA |
| | R | TGTCATCCTTCAGGTCATCAGG |
| NPAS2 | F | ACTCCTCAGCACTAAAGGACAAG |
| | R | GAATGACTGGTATTAAGGCCCGA |
| HFIL3 | F | AAATGCAGACCGTCAAAAAGGAG |
| | R | TCTGACACTTCCGTTAAAGCAGA |
| NR1D1 | F | CTCCAACAACAACACAGGTGG |
| | R | TTGTCACTATAGAGGGATTCAGGG |
| MLL1 | F | CTGGGGAAGCAGACGAACACTA |
| | R | GCAGAATCAAGATCACGCTGGG |
| P300 | F | CTAACATGGGACAGCCGCAAC |
| | R | GAGGCATCATCTGGTTTGGCAC |
| JARID1 | F | CTCCTCCATCAGCTTGTGACCA |
| | R | ACAAACTCCCCAGCACACTGAT |
| SIRT1 | F | CCTCACATGCAAGCTCTAGTGA |
| | R | ATTGTTCGAGGATCTGTGCCAA |
| HDAC3 | F | GCTGGGTGGTGGTGGTTATACT |
| | R | GGAAGCTCCTCACTAATGGCCT |
| EZH2 | F | TCCCAATAACAGTAGCAGGCCC |
| | R | CGTTTCAGTCCCTGCTTCCCTA |
| Beta-2 | F | AGCGTACTCCAAAGATTCAGGTT |
| MICROGLOBULIN | R | GGATGAAACCCAGACACATAGCA |
| Beta-ACTIN | F | GCACCCAGCACAATGAAGATCAAG |
| | R | CACATCTGCTGGAAGGTGGACAG |
| GAPDH | F | GTCAAGGCTGAGAACGGGAA |
| | R | AGCATCGCCCCACTTGATTT |

Statistics and reproducibility. The time of collection was adjusted to Zeitgeber Time (ZT; ZT0 is the equivalent of sunrise at 06:00). All data are presented as ZT time. Graph Pad Prism (version 10) software was used for statistical analysis of data. Data is presented in both spline graph of individual data at various time points and the line graph of mean+SEM values of each group at various time points. Cosinor analysis was used to determine rhythmicity and approximate acrophase values. The significance difference between AE and control group was assessed using unpaired t-test. When two variables (time and treatment or sex and treatment) were compared, 2-way ANOVA followed by Tukey's multiple comparison test were used, with p<0.05 considered significant. In the cosinor model, data of period X units of time is modeled as Y=M+A*cos (2πt/X−Ø).³⁹ In this equation, Y represents the modeled outcome in the units of the circadian variable of interest. M represents the mesor, Ø represents the acrophase, and A represents the curve amplitude.⁴⁰ The results of the 2-way ANOVA analysis are summarized in Table 4.

TABLE 4
Two-Way ANOVA for circadian genes

| Gene | F (DFn, DFd) | P value | F (DFn, DFd) | P value | F (DFn, DFd) | P value |
|---|---|---|---|---|---|---|
| Name | Interaction | Interaction | Time | Time | Group | Group |
| Perl | F (8, 79) = 3.446 | P = 0.0019 | F (8, 79) = 3.974 | P = 0.0005 | F (1, 79) = 171.4 | P < 0.0001 |
| Per2 | F (8, 79) = 5.536 | P < 0.0001 | F (8, 79) = 4.789 | P < 0.0001 | F (1, 79) = 45.61 | P < 0.0001 |
| Per3 | F (8, 79) = 3.440 | P = 0.0019 | F (8, 79) = 1.624 | P = 0.1312 | F (1, 79) = 91.16 | P < 0.0001 |
| Clock | F (8, 79) = 2.306 | P = 0.0282 | F (8, 79) = 0.9328 | P = 0.4945 | F (1, 79) = 23.20 | P < 0.0001 |
| BAML | F (8, 79) = 3.218 | P = 0.0032 | F (8, 79) = 1.300 | P = 0.2557 | F (1, 79) = 36.58 | P < 0.0001 |
| Cry1 | F (8, 79) = 9.740 | P < 0.0001 | F (8, 79) = 10.26 | P < 0.0001 | F (1, 79) = 92.77 | P < 0.0001 |
| Cry2 | F (8, 79) = 10.62 | P < 0.0001 | F (8, 79) = 11.37 | P < 0.0001 | F (1, 79) = 120.5 | P < 0.0001 |
| DBP | F (8, 79) = 5.500 | P < 0.0001 | F (8, 79) = 4.901 | P < 0.0001 | F (1, 79) = 81.52 | P < 0.0001 |
| Dec1 | F (8, 79) = 2.197 | P = 0.0363 | F (8, 79) = 4.970 | P < 0.0001 | F (1, 79) = 49.26 | P < 0.0001 |
| Dec2 | F (8, 79) = 1.815 | P = 0.0864 | F (8, 79) = 2.481 | P = 0.0187 | F (1, 79) = 33.05 | P < 0.0001 |
| NRIDI | F (8, 79) = 2.795 | P = 0.0089 | F (8, 79) = 1.905 | P = 0.0708 | F (1, 79) = 1.194 | P = 0.2778 |
| HLF | F (8, 79) = 0.4751 | P = 0.8703 | F (8, 79) = 2.419 | P = 0.0216 | F (1, 79) = 2.982 | P = 0.0881 |
| NFIL3 | F (8, 79) = 2.063 | P = 0.0494 | F (8, 79) = 0.9361 | P = 0.4919 | F (1, 79) = 26.71 | P < 0.0001 |
| NPAS2 | F (8, 79) = 0.6095 | P = 0.7673 | F (8, 79) = 3.375 | P = 0.0022 | F (1, 79) = 54.29 | P < 0.0001 |
| ARR1B | F (8, 83) = 15.19 | P < 0.0001 | F (8, 83) = 12.56 | P < 0.0001 | F (1, 83) = 200.3 | P < 0.0001 |
| MLL1 | F (8, 83) = 5.711 | P < 0.0001 | F (8, 83) = 2.613 | P = 0.0134 | F (1, 83) = 2.150 | P = 0.1464 |
| P300 | F (8, 83) = 3.316 | P = 0.0025 | F (8, 83) = 2.844 | P = 0.0077 | F (1, 83) = 80.96 | P < 0.0001 |
| SIRT1 | F (8, 83) = 4.739 | P < 0.0001 | F (8, 83) = 3.493 | P = 0.0016 | F (1, 83) = 22.20 | P < 0.0001 |
| EZH2 | F (8, 83) = 4.211 | P = 0.0003 | F (8, 83) = 2.768 | P = 0.0092 | F (1, 83) = 14.62 | P < 0.0003 |
| HDAC3 | F (8, 83) = 4.398 | P = 0.0002 | F (8, 83) = 2.724 | P = 0.0102 | F (1, 83) = 18.01 | P < 0.0001 |
| JARID1 | F (8, 83) = 4.525 | P = 0.0001 | F (8, 83) = 3.447 | P = 0.0018 | F (1, 83) = 41.47 | P < 0.0001 |

### Results

Demographic data of study subjects are presented in Table 5. Children of all sexes, races, and ethnicities between 6:0-10:11 years of age (mean age 8.6 years) with histories of heavy prenatal alcohol exposure (AE; n=23) and typically developing children (CONT; n=26) were assessed. These two subject groups did not significantly differ on age, sex, race, ethnicity, handedness or parental income.

TABLE 5
Demographic information by groups

| | AE | CON | |
|---|---|---|---|
| Demographic Variable | (n = 23) | (n = 26) | p |
| Age in Years [M (SD)] | 8.6 (1.61) | 8.6 (1.71) | .986 |
| Sex [n (% Female)] | 11 (45.8) | 14 (53.8) | .571 |
| Race [n (% White)] | 13 (54.2) | 18 (69.2) | .413 |
| Ethnicity [n (% Hispanic)] | 10 (41.7) | 9 (34.6) | .242 |
| Handedness [n (% Right)] | 19 (79.2) | 23 (88.5) | .370 |
| Family Income [n (%)] | — | — | .442 |
| $10,001-20,000 | 1 (4.2) | 1 (3.8) | |
| $20,001-30,000 | 0 (0) | 2 (7.7) | |
| $30,001-50,000 | 1 (4.2) | 1 (3.8) | |
| $50,001-75,000 | 4 (16.7) | 4 (15.4) | |
| $75,001-100,000 | 4 (16.7) | 1 (3.8) | |
| $100,000+ | 13 (54.2) | 15 (57.7) | |

Note:
Groups included children with heavy prenatal alcohol exposure (AE) and non-exposed controls (CON).

The diurnal changes in the expression levels of core clock genes (BMAL1, CLOCK, PER1,2,3, CRY1,2) in saliva samples of control and AE subjects were compared. The results of these comparisons are summarized in the figures below.

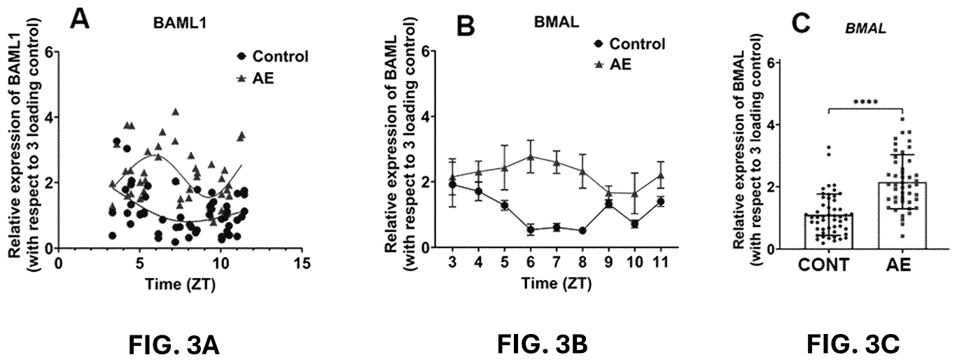

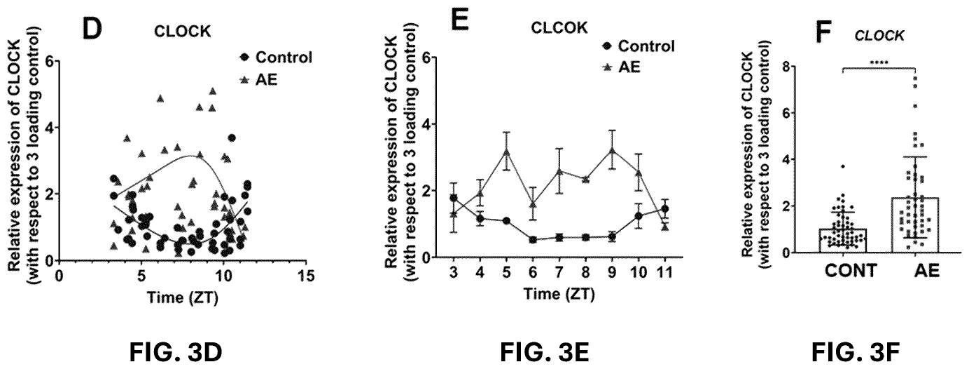

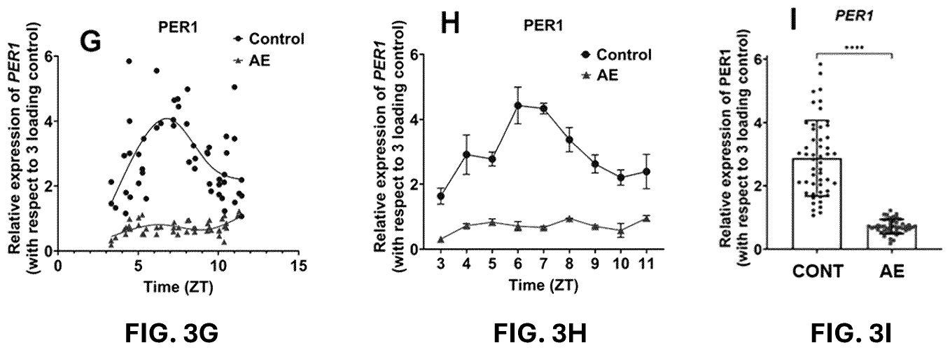

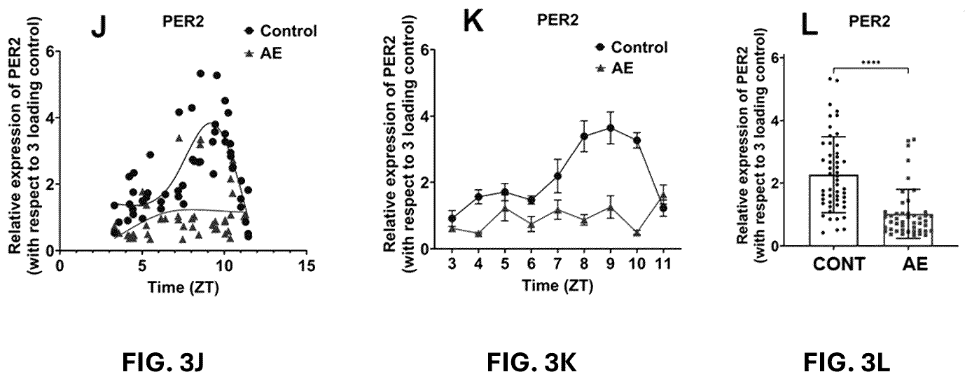

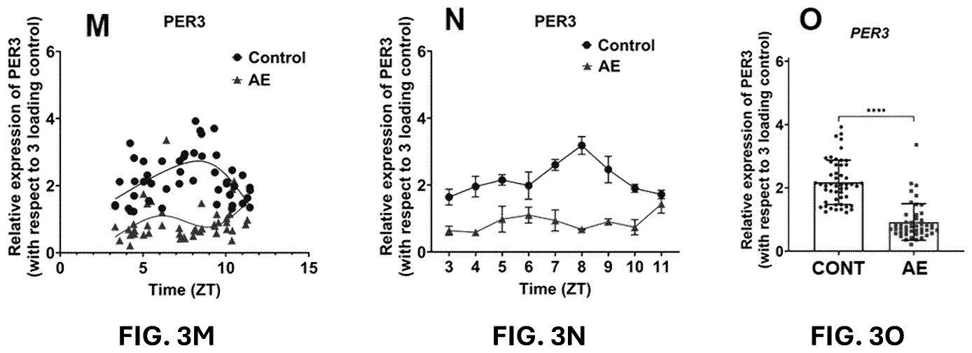

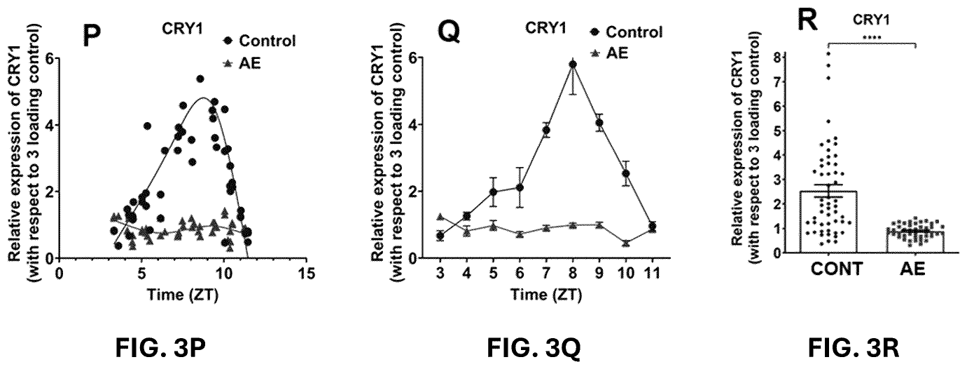

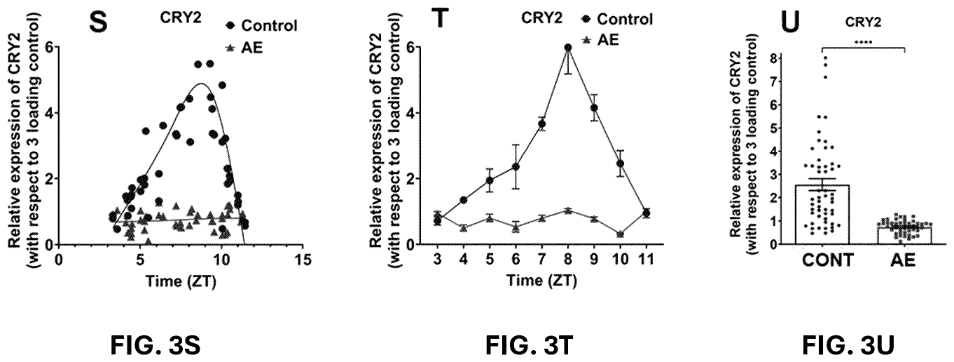

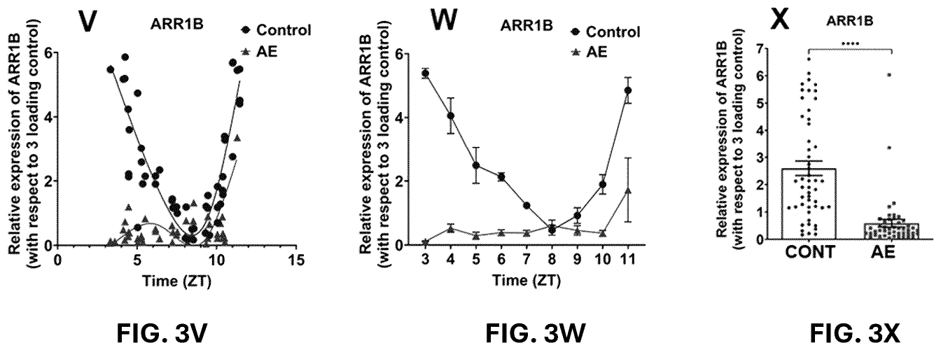

FIGS. 3A-3X shows diagrams describing the effects of prenatal alcohol exposure on circadian expression of core clock genes (FIG. 3A-3U) and a peripheral circadian marker (FIG. 3V-3X). FIG. 3A, FIG. 3D, FIG. 3G, FIG. 3J, FIG. 3M, FIG. 3P, FIG. 3S, and FIG. 3V show changes in the rhythmic expression of the core clock genes BMAL1, CLOCK, PER1, PER2, PER3, CRY1, and CRY2, and the peripheral circadian marker ARR1B, respectively, in salivary samples collected at two time points (morning and afternoon) from each AE and control subjects throughout the day (ZT3 to ZT11) and plotted as spline curves. The diagrams in FIG. 3B, FIG. 3E, FIG. 3H, FIG. 3K, FIG. 3N, FIG. 3Q, FIG. 3T, and FIG. 3W show changes in the expression of the core clock genes BMAL1, CLOCK, PER1, PER2, PER3, CRY1, and CRY2, and the peripheral circadian marker ARR1B, respectively, when plotted as mean±SEM at each hour during ZT 3:00 to ZT 11:00 in AE (ZT3, n=3; ZT4, n=6; ZT5, n=3; ZT6, n=4; ZT7, n=9; ZT8, n=2; ZT9, n=10; ZT, n=3: to ZT11, n=6) and CONT subjects (ZT3, n=3; ZT4, n=7; ZT5, n=6; ZT6, n=3; ZT7, n=5; ZT8, n=6; ZT9, n=5; ZT, n=9: to ZT11, n=7). Data were analyzed using Two-way ANOVA and the interaction between the treatment and time were identified [BMAL1: F (8, 79)=3.218, p=0.0032; CLOCK: F (8, 79)=2.306, p=0.0282; PER1: F (8, 79)=3.446, p=0.0019; PER2: F (8, 79)=5.536, p<0.0001; PER3: F (8, 79)=3.440, p=0.0019; CRY1: F (8, 79)=9.740, p<0.0001; CRY2: F (8, 79)=10.62, p<0.0001; ARR1B: F (8, 83)=16.60, p<0.0001]. FIG. 3C, FIG. 3F, FIG. 3I, FIG. 3L, FIG. 3O, FIG. 3R, FIG. 3U, and FIG. 3X show the overall Mean±SEM levels of gene expression in two groups for the core clock genes BMAL1, CLOCK, PER1, PER2, PER3, CRY1, and CRY2, and the peripheral circadian marker ARR1B, respectively, were analyzed using one-way ANOVA. *** represents a p-value of <0.001.

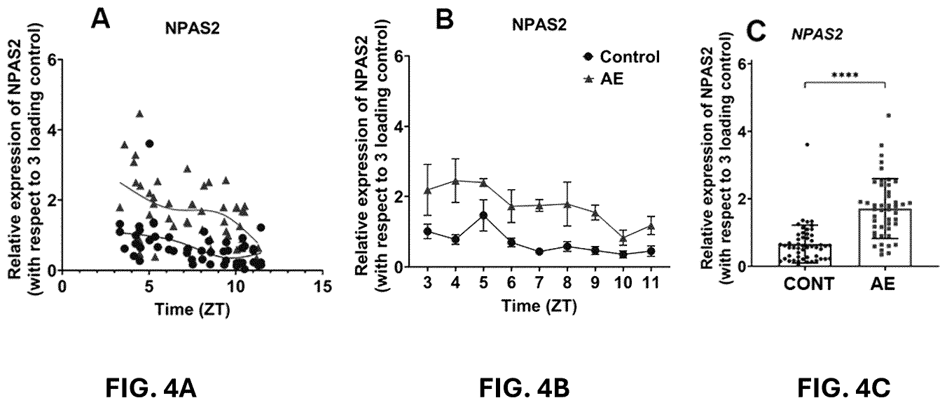

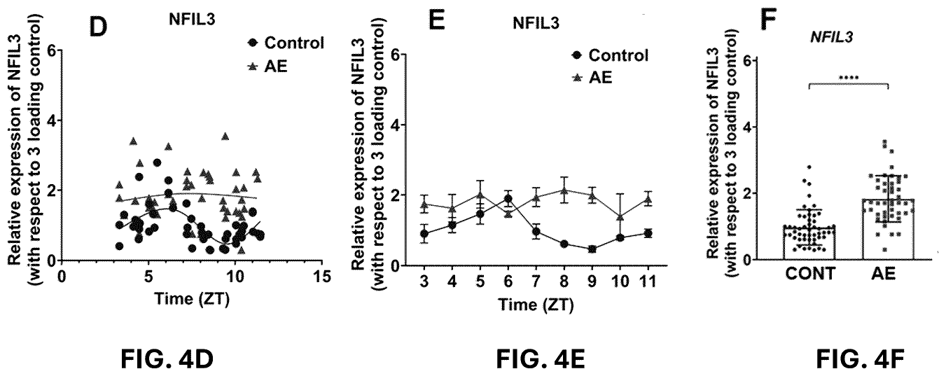

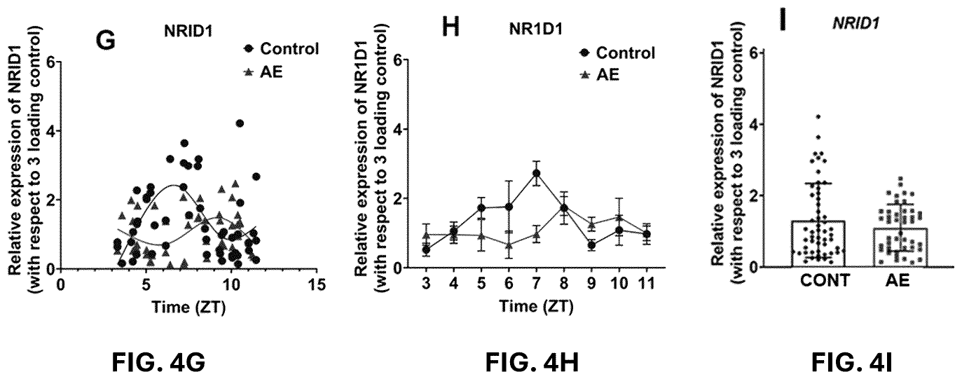

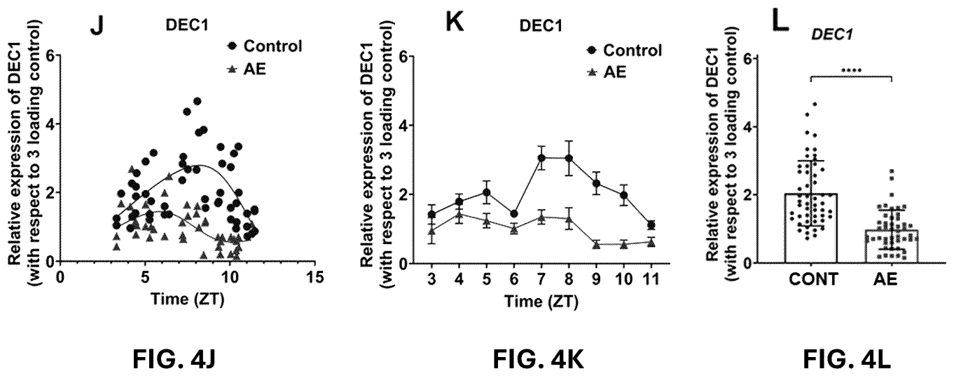

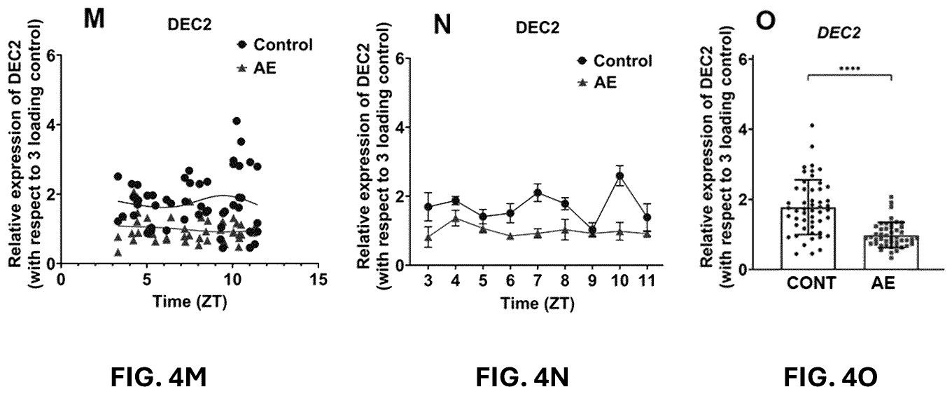

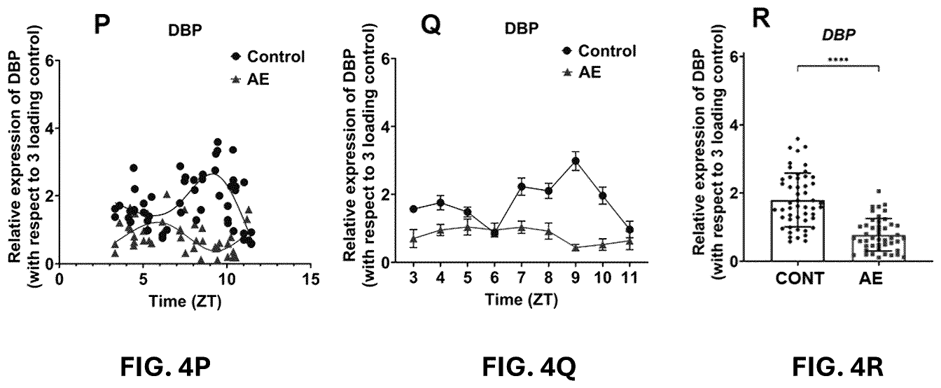

FIGS. 4A-4R shows diagrams describing the effects of prenatal alcohol exposure on circadian expression of an activator NPAS2 (FIG. 4A-FIG. 4C), and repressors (NFL3, FIG. 4D-FIG. 4F; NR1D1, FIG. 4G-FIG. 4I; DEC1, FIG. 4J-FIG. 4L; DEC2, FIG. 4M-FIG. 4O; DBP, FIG. 4P-FIG. 4R) of transcription/translation of core circadian control genes via its role in a negative feedback loop responsible for the control of circadian rhythm. FIG. 4A, FIG. 4D, FIG. 4G, FIG. 4J, FIG. 4M, and FIG. 4P show changes in the rhythmic expression of the activator NPAS2 and the core circadian control genes NFL3, NR1D1, DEC1, DEC2, and DBP, respectively, from each AE and control subjects throughout the day and plotted at the time of collections during ZT 3:00 to ZT 11:00 using spline curves. FIG. 4B, FIG. 4E, FIG. 4H, FIG. 4K, FIG. 4N, and FIG. 4Q changes in the expression of activator NPAS2 and the core circadian control genes NFL3, NR1D1, DEC1, DEC2, and DBP, respectively, when plotted as mean±SEM at each hour during ZT 3:00 to ZT 11:00. The n values are the same as those discussed in FIG. 3. Data were analyzed using Two-way ANOVA and the interaction between the treatment and time were identified [NPAS2: F (8, 79)=0.6095, P=0.7673; NFIL3: F (8, 79)=2.063, P=0.0494; NR1D1: F (8, 79)=2.795, p=0.0089; DEC1: F (8, 79)=2.197, p=0.0363; DEC2: F (8, 79)=1.815, p<0.0864; DBP: F (8, 79)=5.500, p=0.0001]. FIG. 4C, FIG. 4F, FIG. 4I, FIG. 4L, FIG. 4O, and FIG. 4R show the overall Mean±SEM levels of gene expression in two groups for the activator NPAS2 and the core circadian control genes NFL3, NR1D1, DEC1, DEC2, and DBP, respectively, were analyzed using one-way ANOVA. **** represents a p-value of <0.0001.

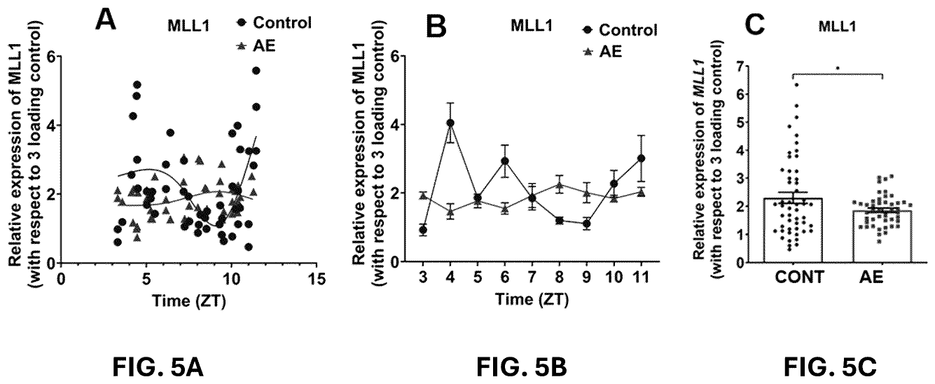

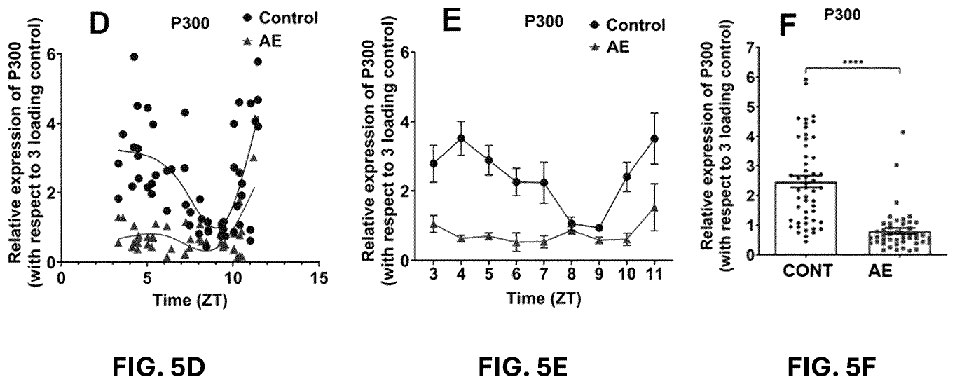

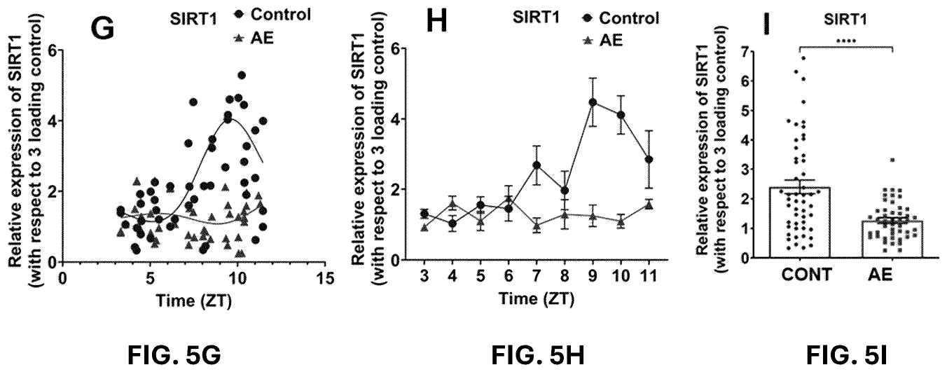

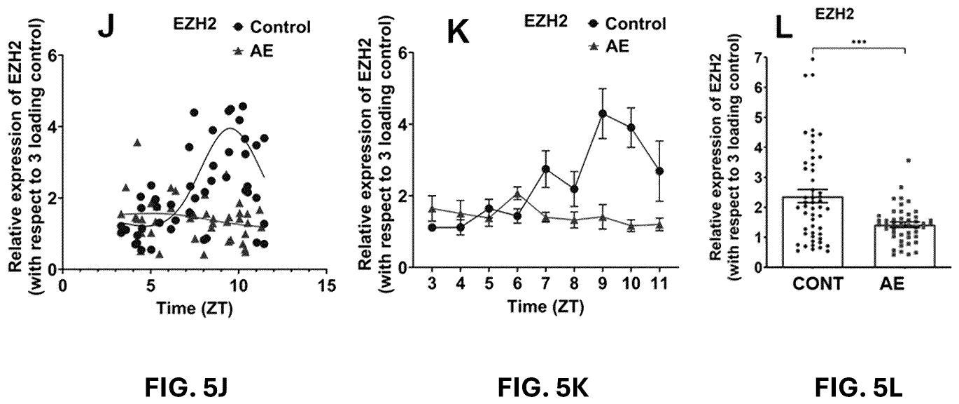

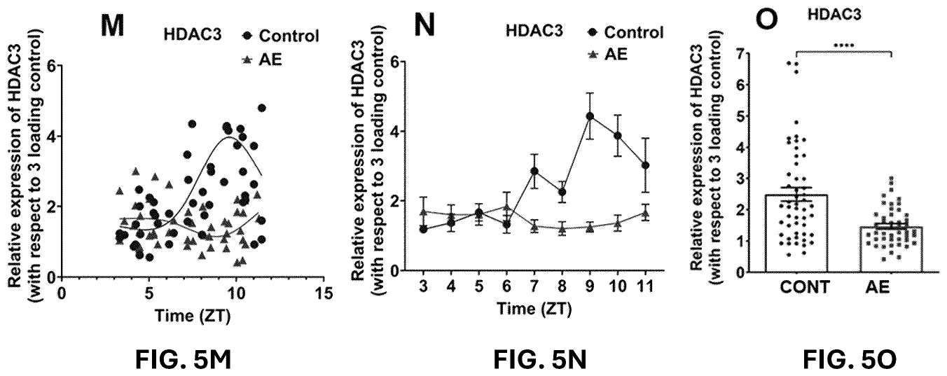

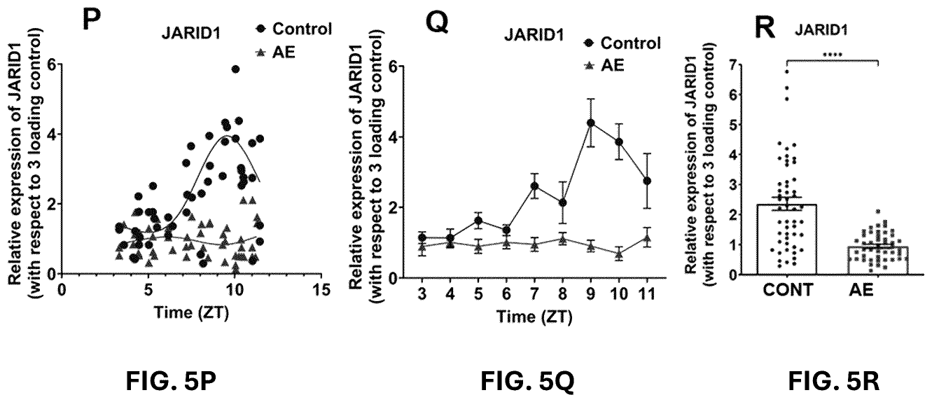

FIGS. 5A-5R shows diagrams describing the effects of prenatal alcohol exposure on circadian expression of various epigenetic modulators known to interact with CLOCK:BMAL1 and impose oscillatory patterns to the epigenetic mark at the promoter of PERICRY gene to maintain circadian rhythmicity, such as the transcription activators MLL1 (FIG. 5A-FIG. 5C) and P300 (FIG. 5D-FIG. 5F); and the transcription inhibitors SIRT1 (FIG. 5G-FIG. 5I), EZH2 (FIG. 5J-FIG. 5L), HDAC3 (FIG. 5M-FIG. 5O), and JARID1 (FIG. 5P-FIG. 5R). FIG. 5A, FIG. 5D, FIG. 5G, FIG. 5J, FIG. 5M, FIG. 5P show changes in the rhythmic expression of the epigenetic modulators of core clock genes MLL1, P300, SIRT1, EZH2, HDAC3, and JARID1, respectively, in salivary samples collected at two time points (morning and afternoon) from each AE and control subjects throughout the day and plotted at the time of collections during ZT 3:00 to ZT 11:00. FIG. 5B, FIG. 5E, FIG. 5H, FIG. 5K, FIG. 5N, and FIG. 5Q show changes in the expression of the epigenetic modulators of core clock genes MLL1, P300, SIRT1, EZH2, HDAC3, and JARID1, respectively, when plotted as mean±SEM at each hour during ZT 3:00 to ZT 11:00. The n values are the same as those discussed in FIG. 3. Data were analyzed using Two-way ANOVA and the interaction between the treatment and time were identified [MLL1: F (8, 83)=5.711, P=0.0001; P300: F (8, 83)=3.316, P=0.0025; SIRT1: F (8, 83)=4.739, p=0.0001; EZH2: F (8, 83)=4.211, p=0.0003; HDAC3: F (8, 83)=4.398, p<0.0002; JARID1: F (8, 83)=4.525, p=0.0001]. FIG. 5C, FIG. 5F, FIG. 5I, FIG. 5L, FIG. 5O, and FIG. 5R show the Overall Mean±SEM levels of gene expression in two treatment groups were analyzed using one-way ANOVA. * represents a p-value of <0.5; *** represents a p-value of <0.001; **** represents a p-value of <0.0001.

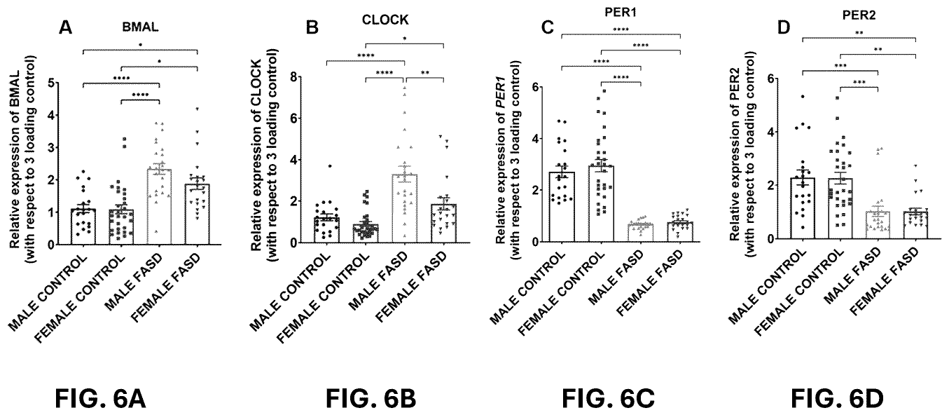

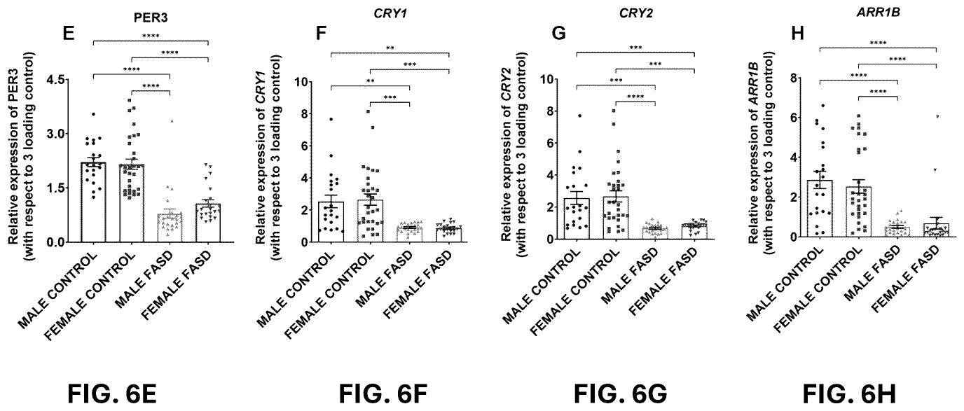

FIGS. 6A-6H shows diagrams describing the changes in the Mean+SEM levels of core clock genes (FIG. 6A: BMAL; FIG. 6B: CLOCK; FIG. 6C: PER1; FIG. 6D: PER2; FIG. 6E: PER3; FIG. 6F: CRY1; FIG. 6G: CRY2) and peripheral circadian marker ARR1B (FIG. 6H) in male and female control and FASD (prenatal ethanol exposed) children (n; Male—Control, 21; −FASD, 24; Female—Control, 30; FASD, 22) . Data were analyzed using one-way ANOVA. * represents a p-value of <0.05; ** represents a p-value of <0.01; *** represents a p-value of <0.001, **** represents a p-value of <0.0001.

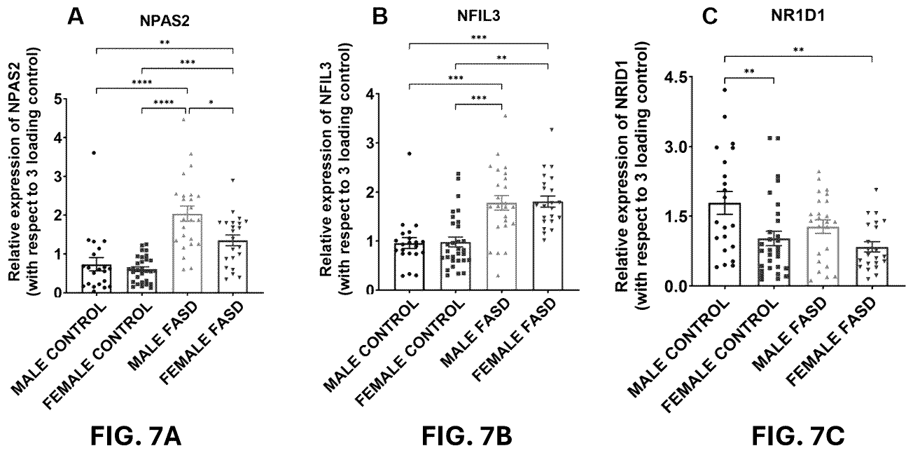

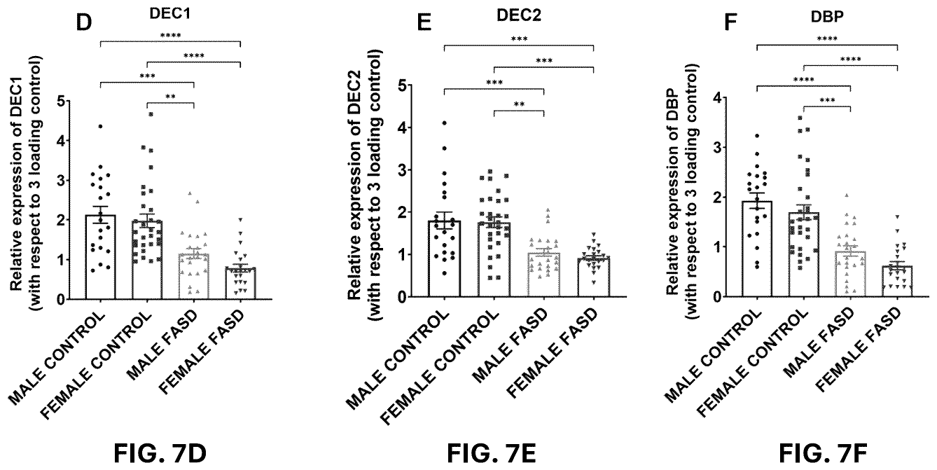

FIGS. 7A-7F shows diagrams describing the changes in the Mean+SEM levels of an activator NPAS2 (FIG. 7A), and repressors NFL3 (FIG. 7B), NR1D1 (FIG. 7C), DEC1 (FIG. 7D), DEC2 (FIG. 7E), and DBP (FIG. 7F) of transcription/translation of core circadian control genes in male and female control and FASD (prenatal ethanol exposed) children (n; Male—Control, 21; −FASD, 24; Female—Control, 30; FASD, 22) . Data were analyzed using one-way ANOVA. * represents a p-value of <0.05; ** represents a p-value of <0.01; *** represents a p-value of <0.001; **** represents a p-value of <0.0001.

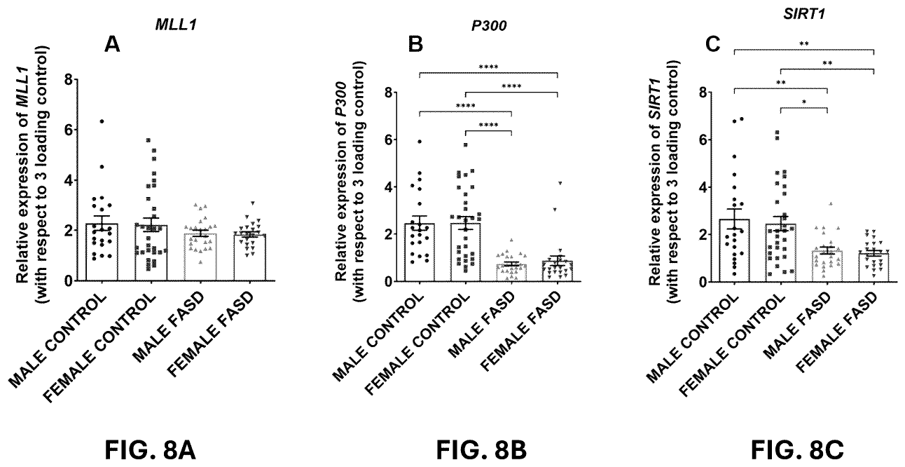

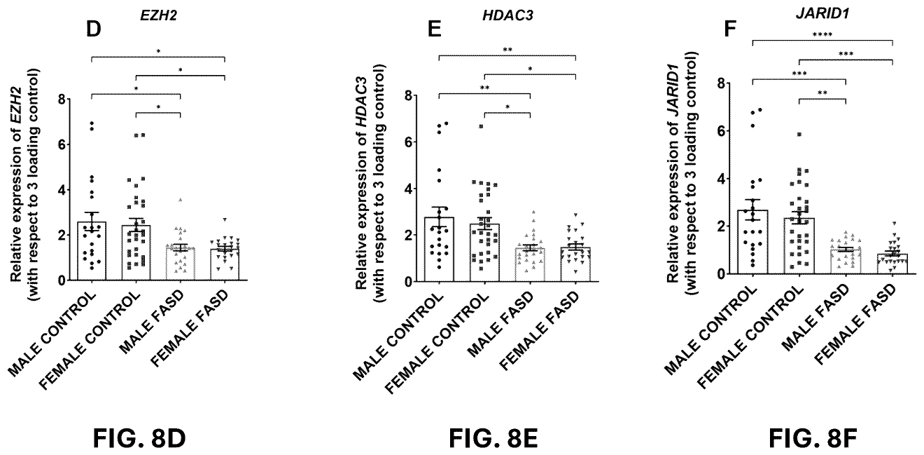

FIGS. 8A-8F shows diagrams describing the changes in the Mean+SEM levels of various epigenetic modulators known to interacts with CLOCK:BMAL1 and imposes oscillatory pattern to the epigenetic mark at the promoter of PER/CRY gene to maintain circadian rhythmicity in in male and female control and FASD (prenatal ethanol exposed) children (n; Male—Control, 21; −FASD, 24; Female—Control, 30; FASD, 22), including the transcription activators MLL1 (FIG. 8A) and P300 (FIG. 8B), and the transcription inhibitors SRT1 (FIG. 8C), EZH2 (FIG. 8D), HDAC3 (FIG. 8E), and JARID1 (FIG. 8F). Data were analyzed using one-way ANOVA. * represents a p-value of <0.05; ** represents a p-value of <0.01; *** represents a p-value of <0.001, **** represents a p-value of <0.0001.

The data the present examples shows that in control subjects, expression patterns (as determined by spline graph of individual data at various time points and the line graph of mean+SEM values of each group at various time points) of primary circadian activator genes BMAL1 (see FIGS. 3A-3B) and CLOCK (see FIGS. 3D-3E) fluctuated throughout the day and showed lower expression levels in the afternoon. Cosinor analysis of expression data identified a circadian nature of the rhythm (see FIG. 3Y; Zero-amplitude test p values: BMAL1, 0.008, CLOCK, 0.0001; Acrophase times for BMAL1, ZT20.46.83h; CLOCK, ZT19.49h). In subjects with AE, the gene expression patterns of these two activators BMAL1 and CLOCK were elevated and circadian expression patterns were altered (Zero-amplitude test p values: BMAL1, 0.0.311; CLOCK, 0.0906; acrophase times: BMAL1, ZT4.42.4h; CLOCK, ZT7.27). The mean levels of the Bmal1 and Clock genes at various time points (see FIG. 3B, FIG. 3F; see also Table 4) and overall mean levels (see FIG. 3C) were higher (with a p-value of <0.001) in the AE group than those in the control group.

The overall mean expression of BMAL1 did not show any sex difference in AE effects [F (1, 93)=1.972, p=0.1636], while CLOCK1 expression did show significant sex differences, with females showing reduced expression compared to males [F (1, 93)=10.60, p=0.0016] (FIG. 6B-6C). In the case of primary repressor genes of circadian clock in CONT group, we found that all PER genes (PER1, FIG. 3G-3H: PER2, FIG. 3J-3K; PER3, FIG. 3M-3N) and CRY genes (CRY1, FIG. 3P-3Q; CRY2, FIG. 3S-3T) showed rhythmic and elevated expression during the late morning to early afternoon in CONT children. A cosinor analysis of expression data identified the circadian nature of the rhythm. Zero-amplitude test p values for PER1, 0.00048; PER2, 0.00001; PER3, 0.00014; CRY1, 0.00001: CRY2, 0.00001) with acrophase times as follows in control subjects (PER1, ZT07.26; PER2, ZT8.36; PER3, ZT7.55; CRY1, ZT7.83: CRY2, ZT7.80). The results of this cosinor analysis are summarized on Table 6 below.

TABLE 6
Cosinor analysis to determine the circadian rhythm of
core clock genes and peripheral circadian marker.

| Gene | | Acrophase | Zero-amplitude |
|---|---|---|---|
| name | Treatment | (ZT ours) | test (p value) |
| BMAL | Control | 20.46 | p = 0.00874 (s) |
| | AE | 4.42 | p = 0.31104 (ns) |
| CLOCK | Control | 19.49 | p = 0.0001 (s) |
| | AE | 7.27 | p = 0.0906 (ns) |
| PER1 | Control | 7.26 | p = 0.00048 (s) |
| | AE | 10.84 | p = 0.09267 (ns) |
| PER2 | Control | 8.36 | p = 0.00001 (s) |
| | AE | 11.68 | p = 0.09403 (ns) |
| PER3 | Control | 7.55 | p = 0.00014 (s) |
| | AE | 14.93 | p = 0.2263 (ns) |
| CRY1 | Control | 7.83 | p = 0.00001 (s) |
| | AE | 21.54 | p = 0.49685 (ns) |
| CRY2 | Control | 7.80 | p = 0.00001 (s) |
| | AE | 17.34 | p = 0.41161 (ns) |
| ARR1B | Control | 19.80 | p < 0.00001 (s) |
| | AE | 9.36 | p = 0.52837 (ns) |

s = significant;
ns = nonsignificant

However, in subjects with AE, all PER and CRY gene expression were reduced and circadian oscillations were disrupted (Cosinor p values; PER1, 0.09267; PER2, <0.09403; PER3, 0.0.2263; CRY1, 0.49685: CRY2, 0.41161) and acrophase times were modified (PER1, ZT10.84; PER2, ZT11.68; PER3, ZT14.93; CRY1, ZT21.54: CRY2, ZT17.34). The mean levels of PER1-3 and CRY1-2 genes at various time points (FIG. 3H, 3K, 3N, 3P, 3S and Table 4) and overall mean levels (FIG. 3I, 3L, 30, 3R, 3U) were lower (p<0.001) in AE groups than those in the control groups. The overall mean levels of these genes did not show any sex differences [PER1: F (1, 93)=0.1922, p=0.6621; PER2: F (1, 93)=0.06520, p=0.7990; PER3: F (1, 93)=0.3453, p=0.5582; CRY1: F (1, 93)=0.06445, p=0.8001; CRY2: F (1, 93)=0.04588, p=0.8309] (FIG. 6D-6H).

In the present examples, the expression pattern of ARRB1 was also determined. ARRB1 is considered a molecular marker of the peripheral circadian rhythm.¹³ ARRB1 levels in saliva of control subjects fluctuated throughout the day in a rhythmic fashion and showed lower expression levels in the afternoon, as determined by spline graph of individual data at various time points (FIG. 3V) and the line graph of mean+SEM values of each group at various time points (FIG. 3W). Cosinor analysis of expression data identified the circadian nature of the rhythm (Table 6; Zero-amplitude test p value <0.0001) with an acrophase value of ZT19.80. However, ARRB1 levels in saliva of subjects with AE were markedly reduced and fluctuated without a clear circadian pattern (Zero-amplitude test p value 0.5283) with an acrophase time of ZT9.36. The overall mean level of ARRB1 was lower (p<0.0001) in AE group compared to control group (FIG. 3X). Also, there were no significant sex differences [F (1, 93)=0.4623, p=0.4983] in the mean expression level of ARRB1 in AE and control groups (FIG. 6A). These data suggest that AE affects the daily circadian oscillation, which is maintained by a coordinated transcription-translation feedback loop between the core clock activators (BMAL and CLOCK) and core clock repressors (PER and CRY) (Table 6).

The salivary expression patterns of an activator NPAS2 were also determined, along with those of several repressors (NFL3, NR1D1, DEC1, DEC2, DBP) of transcription/translation of core circadian control genes, as they are involved in a negative feedback loop responsible for the control of circadian rhythm.⁴³ The present data revealed that in control subjects, expression patterns (as determined by spline graph of individual data at various time points and the line graph of mean+SEM values of each group at various time points) of NAPS2 (FIG. 4A-4B) and NFL3 (FIG. 4D-4E) fluctuated throughout the day. The NPAS2 gene expression pattern showed circadian rhythm (Cosinor p=0.0021) with the estimated acrophase time is at ZT23.5 in the control subjects and it remained rhythmic (Cosinor p-value of <0.006) with the estimated acrophase time is at ZT1.70 in subjects with AE. The results for this cosinor analysis are summarized in Table 7.

TABLE 7
Cosinor analysis to determine the circadian rhythm
of core clock genes' activator (NPAS2) and
repressors (NFL3, NR1D1, DEC1, DEC2, DBP).

| | | Acrophase | Zero amplitude |
|---|---|---|---|
| Gene name | Treatment | (ZT hours) | test (p value) |
| NPAS2 | Control | 23.50 | p < 0.00218 (s) |
| | AE | 1.70 | p = 0.00609 (s) |
| NFL3 | Control | 21.64 | p = 0.1733 (ns) |
| | AE | 8.45 | p = 0.86879 (ns) |
| NR1D1 | Control | 7.24 | p = 0.1463 (ns) |
| | AE | 15.58 | p = 0.16733 (ns) |
| DEC1 | Control | 7.48 | p = 0.0001 (s) |
| | AE | 4.56 | p = 0.00127 (s) |
| DEC2 | Control | 8.32 | p = 0.82459 (ns) |
| | AE | 1.16 | p = 0.45444 (ns) |
| DBP | Control | 7.78 | p = 0.02124 (s) |
| | AE | 4.90 | p = 0.03293(s) |

s = significant;
ns = nonsignificant

A zero-amplitude test p-value of <0.05 identifies rhythmicity, and the acrophase value shows the peak time of the rhythm in ZT hours. The mean levels of NPAS2 genes at various time points (FIG. 4B; see Table 4) or overall mean levels (FIG. 4C) were higher (p<0.001) in AE group than those in the control group, without showing any sex differences in the AE response [F (1, 87)=3.469, p=0.0659] (FIG. 7A). The levels of NFL3, NR1D1, and DEC2 in saliva showed no rhythmicity in control (Table 7; Cosinor p=; NFL3-0.1733, NR1D1-0.1463, and DEC2-0.8245) or AE subjects (FIG. 2S; Cosinor p=; NFL3-0.863, NR1D1-0.1673, and DEC2-0.4544), but had acrophase modification (FIG. 2S; control-NFL3-ZT21.64, NR1D1-ZT7.24, and DEC2-ZT8.32; AE-NFL3-ZT8.45, NR1D1-ZT15.58, and DEC2-ZT1.16) and mean level elevation (p<0.001) at various time points (FIG. 4B; see Table 4) or overall mean levels (FIG. 4C). Other repressors of core circadian control genes (DEC1 and DBP) were expressed in a circadian manner in both control (Cosinor p; DEC1-0.0001, DBP-0.0212) and AE subjects (Cosinor p; DEC1-0.0012, DBP-0.0329) in saliva samples of control subjects; however, acyclic expression in some but not all of these genes were seen (Cosinor p; NR1D1-0.1562 DEC1-0.0004 DEC2-0.445; DBP-0.0001) in AE subjects (FIG. 4G, FIG. 4J, FIG. 4M, FIG. 4P). The estimated acrophase times of these genes also differed between Control (NR1D1-ZT 7.22, DEC1-7.45h, DEC2-ZT8.139h, DBP-ZT7.78h,) and AE subjects (NR1D1-ZT 16,23h, DEC1-ZT 4.937, DEC2 ZT 1.201, DBP-ZT 5.5). The mean levels of DEC1, DEC2, DBP genes but not NR1D1 genes at various time points (FIG. 4H, FIG. 4K, FIG. 4N, FIG. 4Q; see Table 4) or overall mean levels of DEC1, DEC2, DBP genes but not NR1D1 were also lower (p<0.0001) in the AE group compared to those in the control group (FIG. 41, FIG. 4L, FIG. 4O, FIG. 4R). The responses of these clock regulatory genes did not show any sex differences [NPAS2: F (1, 87)=3.469, p=0.0659; NFL3: F (1, 89)=0.009085, p=0.9243; NR1D1: F (1, 85)=8.675, p=0.0042; DEC1: F (1, 93)=3.280, p=0.0733; DEC2: F (1, 92)=1.039, p=0.3018; DBP: F (1, 93)=5.681, p=0.0192] (FIG. 7B-F). These data suggest that AE effects may involve an alteration in the CLOCK/BMAL1 complex, which is regulated through epigenetic modifications via various activators and repressors at the CRY and PER promoters to influence their circadian expression (Table 7).

Prenatal alcohol exposure is known to impact the body's epigenome to modulate the global transcriptome.^{44,45} There are reports that epigenetic modulators interact with the CLOCK:BMAL1 complex and impose an oscillatory pattern to the epigenetic mark at the promoter of PER/CRY gene to maintain circadian rhythmicity.¹⁸ Expression patterns of MLL1 and P300 genes, which are known to interact with CLOCK:BMAL1 and are recruited in the CCG promoters in a circadian fashion,⁴⁶ are rhythmic. This is revealed by the finding that in control subjects, expression patterns (as determined by spline graph of individual data at various time points and the line graph of mean+SEM values of each group at various time points) of MLL1 (FIG. 5A-FIG. 5B) and P300 (FIG. 5D-FIG. 5E) fluctuated throughout the day. The MLL1 gene expression pattern showed circadian rhythm (Cosinor p; MLL1, 0.0482; P300, 0.0003) and had estimated acrophase times in late night (MLL1, ZT 19.895; P300, ZT 19.830) in control subjects, like BMAL1 and CLOCK (see FIG. 4).

In AE subjects, the expression pattern of these epigenetic modifiers was arhythmic (Cosinor p; MLL1, 0.362; P300, 0.524), and estimated acrophase times were advanced (MLL1, ZT10.662; P300, ZT20.149) (FIG. 5B. FIG. 5E, FIG. 5S). The mean levels of MLL1 gene and P300 gene at various time points (FIG. 5B, FIG. 5E; FIG. 4) or overall mean levels were lower (p<0.05) in AE group than those in the control group (FIG. 5C, FIG. 5F). Expression patterns of other epigenetic modulators (SIRT1, EZH2, HDAC3, JARID1) known to favor a repressive state of chromatin and be involved in repression of PER/CRY gene transcription⁴⁷⁻⁴⁸ were also measured. In control subjects, expression patterns of these repressors (FIG. 5G, FIG. 5J, FIG. 5M, FIG. 5P) were rhythmic (Cosinor p; SIRT1, 0001; EZH2, 0.0002; HDAC3, 0.0001; JARID1, 0.0001) and had estimated acrophase times in late afternoon (SIRT1, ZT10.6; EZH2, ZT9.819; HDAC3, ZT10.398; JARID1, ZT10.126), whereas in AE subjects, expression patterns these genes were arhythmic (Cosinor p; =SIRT1, 0.899; EZH2, 0.264; HDAC3, 0.276; JARID1, 0.665), and their estimated acrophase times were altered (SIRT1, ZT21.561; EZH2, ZT5.113; HDAC3, ZT21.266; JARID1, ZT5.788). The mean levels of all CLOCK regulatory epigenetic genes at various time points (FIG. 5H, FIG. 5K, FIG. 5N, FIG. 5Q; see Table 4) and the overall mean levels of these genes were lower (p<0.0001) in AE group than those in the control group (FIG. 5I, FIG. 5L, FIG. 5O, FIG. 5R). No sex differences were found in the response to AE in these epigenetic genes [MLL1: F (1, 93)=0.1136, p=0.736; NFL3: F (1, 89)=0.009085, p=0.9243; P300: F (1, 93)=0.02040, p=0.5876; SRT1: F (1, 93)=0.4435, p=0.5071); EZH2: F (1, 93)=0.2548, p=0.614; HDAC3: F (1, 93)=0.4185, p=0.5193; JARID1: F (1, 93)=1.513, p=0.2218] (FIG. 8A-FIG. 8F). These data suggest that AE may also mediate alterations in the CLOCK/BMAL1 complex-regulated epigenetic machinery which involves activators and repressors at the CRY and PER promoters to influence their circadian rhythmic expression (Table 8).

TABLE 8
Cosinor analysis to determine the circadian rhythm
of epigenetic modulators (transcription activators
and inhibitors) of core clock genes

| Gene | | Acrophase | Zero-amplitude |
|---|---|---|---|
| Name | Treatment | (ZT hours) | Test (p value) |
| MLL1 | Control | 19.895 | p < 0.0482 (s) |
| | AE | 10.662 | p = 0.3623 (ns) |
| P300 | Control | 19.830 | p = 0.0003 s) |
| | AE | 20.149 | p = 0.5244 (ns) |
| SIRT1 | Control | 10.606 | p = 0.0001 (s) |
| | AE | 21.561 | p = 0.8999 (ns) |
| EZH2 | Control | 9.819 | p = 0.0002 (s) |
| | AE | 5.113 | p = 0.2649 (ns) |
| HDAC3 | Control | 10.398 | p = 0.0001 (ns) |
| | AE | 21.266 | p = 0.2769 (ns) |
| JAR1D1 | Control | 10.128 | p = 0.0001 (s) |
| | AE | 5.788 | p = 0.6653 (ns) |

s = significant;
ns = nonsignificant

A zero-amplitude test p-value of <0.05 identifies rhythmicity, and the acrophase value shows the peak time of the rhythm in ZT hours. Data presented here are consistent with the previous finding that CLOCK genes are expressed in a circadian manner in oral mucosa in healthy diurnally active volunteers.⁴² PER genes peaks during early afternoon around ZT 07:00-08:00 while BMAL1 expression peaks in late night around ZT 18:00-19:00.^{26,49-51} The peak times for PER1 and CRY1 by the cosinor curve fit are similar to those found in human oral mucosa,⁴² and in the SCN and the peripheral tissues in rodents.⁵²⁻⁵³ Furthermore, the ARRB1 gene, known to be a candidate biomarker of peripheral circadian rhythm,¹³ also showed rhythmic expression in saliva in the control subjects. The study of the present example also revealed a loss of rhythmicity in all core clock gene transcripts, as well as ARRB1, in saliva samples of subjects with AE, suggesting that alcohol exposure during the prenatal period adversely affects the circadian expression of core clock genes.

The study of the present example also identified significant changes in overall transcript levels of core clock genes in subjects with symptoms of FASD. The overall mean levels of PERs and CRYs were lower, whereas BMAL1 and CLOCK genes were higher in the AE group, suggesting that AE may act on the circadian positive feedback loop involving BMAL1 and CLOCK heterodimers effects on transcriptions of PER and CRY genes. Thus, AE affects the daily circadian oscillation maintained by a coordinated transcription-translation feedback loop between the core clock activators (BMAL and CLOCK) and core clock repressors (PER and CRY) (Table 6).

Alcohol exposures during the prenatal period also affects the expression patterns of various regulators of core clock genes. In the core negative feedback loop, NAPS2 acts as an activator while NFL3, NR1D1, DEC1, DEC2, and DBP serve as repressors of transcription/translation of core circadian control genes and its circadian rhythm. These proteins are also shown to inhibit CLOCK-BMAL1-mediated transcription of E-box-containing promoters. Additional regulators controlling this circuitry include activator DBP and the repressor NFIL3 through the D-box element. Prenatal alcohol exposure affects both the circadian patterns and the expression levels of these repressor genes (Table 7). These data suggest that AE may act on multiple components of the circadian feedback loop to impair typical circadian oscillation.

These data also determined the role of epigenetic modulation of circadian genes in FASD. The CLOCK:BMAL1 heterodimer activates the transcription of several CCGs through their binding to the E-Box elements in their promoters and the recruitment of coactivating complexes such as P300 that acetylates the lysine residues of the histones allowing euchromatin formation.⁴⁶ The MLL1 methyltransferase responsible for methylation in H3K4 (mark related to transcriptional activation) is associated with CLOCK and is recruited in the CCG promoters in a circadian manner to promote transcription.⁴⁶ In addition to the repression mediated by PER, CRY, histone deacetylation mediated by histone deacetylases like SIRT1 and HDAC3 play a role to favor a repressive state of chromatin. Thus, AE effects may involve alteration in the CLOCKC/BMAL-1 complex-regulated epigenetic modifications by various activators and repressors at the CRY and PER (and other clock regulatory genes) promoters to influence their circadian expression.

In summary, these data show that the daily circadian oscillation which is maintained by a coordinated transcription-translation feedback loop between the core clock activators (BMAL and CLOCK) and core clock repressors (PER and CRY) is affected by AE. The daily circadian oscillation is maintained by a coordinated transcription-translation feedback loop between the core clock activators (BMAL and CLOCK, or BMAL and NAPS2) and core clock repressors (PER and CRY). The other negative regulators of the molecular clockwork, DEC proteins, are also shown to inhibit CLOCK-BMAL1-mediated transcription of E-box-containing promoters. Also, NR1D and ROR proteins, which compete to bind RORE to alter the expression of various circadian clock genes (CCGs), including BMAL. Additional regulators controlling this circuitry include activator DBP and HLF and the repressor NFIL3 through the D-box element. Prenatal alcohol exposure affects various sites of this circadian feedback loop to disrupt the daily circadian oscillation. The effects may involve alterations in CLOCK/BMAL1 complex regulated epigenetic modifications by various activators and repressors at the CRY and PER (other clock regulatory genes) promoters to influence their circadian expression.

Importantly, there is a strong association between disruption of circadian rhythms and psychiatric illnesses including sleep disorders, anxiety behavior and hyperreactive stress responses often observed in children with FASD.⁵⁴⁻⁵⁹ Thus, circadian clock dysfunction may represent both a marker for pathology and a crucial opportunity for early intervention in FASD. Hence, detection of circadian abnormalities and restoration of circadian rhythms in preclinical FASD or childhood diseases may provide an opportunity for early intervention to alter the developmental trajectory and quality of life.

### Example 2

### Experimental Details—AE in Rat Pituitary Cells

DPPA4 is shown to be overexpressed in AE-exposed human placenta,⁶⁰ the pituitary of adult AE rats, and pituitary of human surgical patients.⁶¹⁻⁶³ Additionally, the data of GEO base (GSE120952) indicates that CRISPR-Cas9 mediated DPPA4 knockdown of mouse embryonic cells affects the expression of core clock genes. A quantified RNAseq report of this data is shown in Table 9.

TABLE 9
GSE120952_Quantified_RNAseq_report_KO

| Gene name | CRISPR_c23_Dppa4_KO | CRISPR_c29_Dppa4_KO | CRISPR_c57_WT | CRISPR_c58_WT |
|---|---|---|---|---|
| Per1 | 4.904413 | 4.789953 | 4.234753 | 4.3687515 |
| Amtl | 2.9139316 | 3.0509763 | 3.4826255 | 3.5369406 |
| Cry1 | 5.5326586 | 5.4680524 | 5.0226755 | 4.407015 |

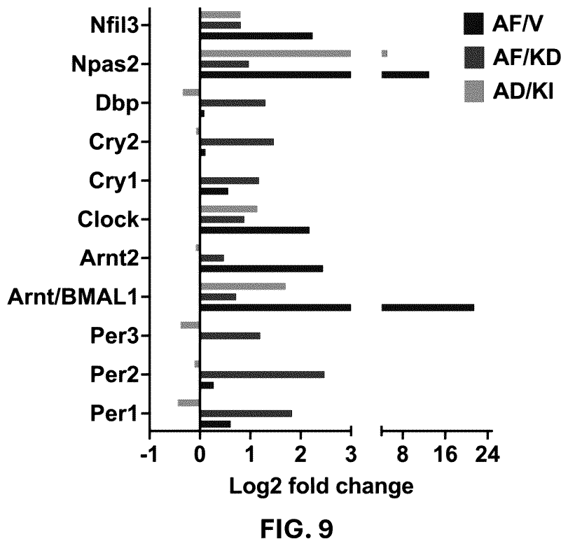

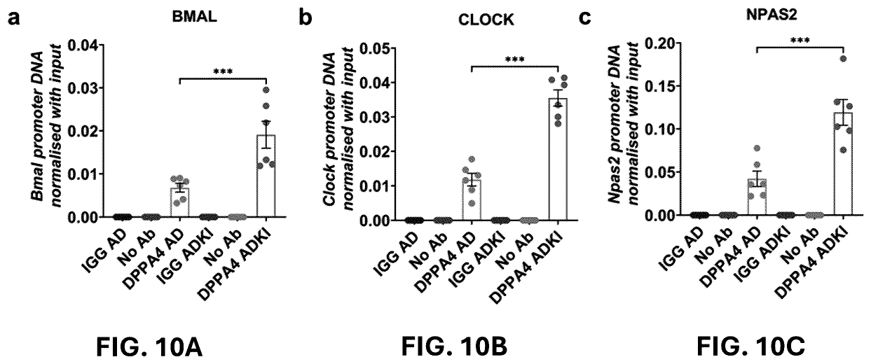

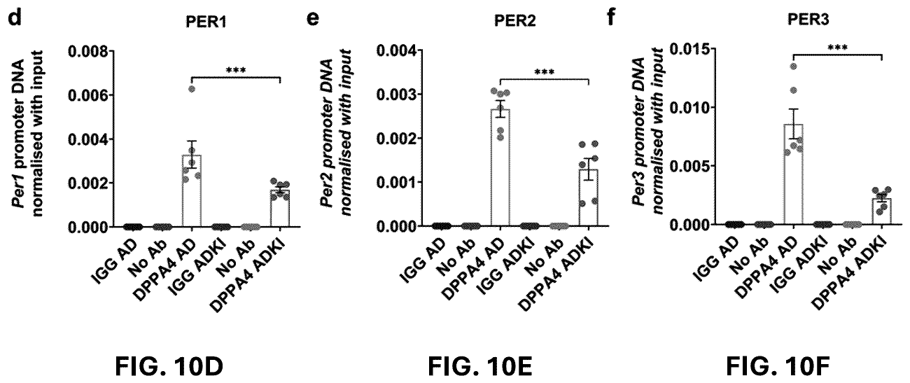

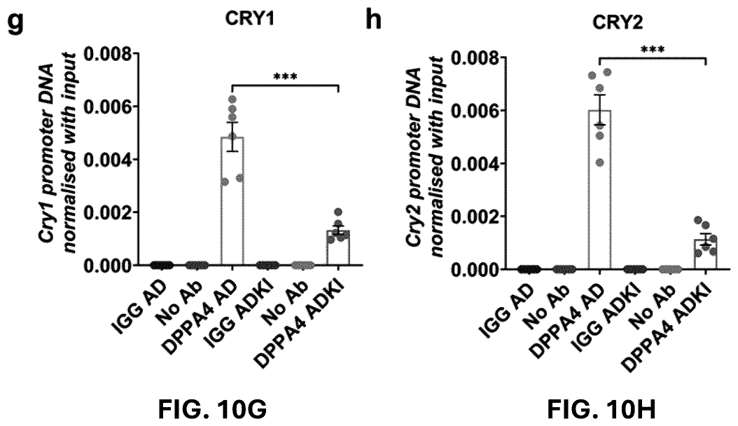

Using AE rat pituitary cells,⁶⁵ DPPA4 gene expression was modified by CRISPR knockdown and knock-in methods. Cells were harvested and used for RNA-seq and ChIP analysis. Procedures for each of these methods are described below. RNA-seq data show that alcohol-fed AF group (Dppa4 high) had high levels of Bmal1, Clock, Npas2, Nfil3 and low levels of Per1, Per2, Per3, Cry1, Cry2. Knockdown of Dppa4 in AF cells (AFKD) reversed the whole expression pattern, while knockdown of Dppa4 in AD (non-alcohol fed group) mimicked the expression pattern. FIG. 9 shows a comparison plot of AF/V (alcohol fed-vector treated), AFKD (AF-Dppa4 knockdown), and ADKI (control-fed-DPPA4 over expressed) with respect to AD/V (control fed-vector treated) shows the effect of DPPA4 on the expression of different circadian genes. Data were obtained from RNA-seq of rat pituitary cells, with n=3. ChIP analysis indicated that DPPA4 binding on BMAL, CLOCK and NAPS2 are increased while PER1-3 and CRY1,2 decreased following DPPA4 knock-in in control pituitary cells. FIGS. 10A-10H shows comparison plots which demonstrate that the promoters of different circadian gene are regulated by DPPA4. ChIP assays data show the binding of DPPA4 in the promoter region of (FIG. 10A) BMAL, (FIG. 10B) CLOCK, (FIG. 10C) NPAS2, (FIG. 10D) PER1, (FIG. 10E) PER2, (FIG. 10F) PER3, (FIG. 10G) CRY1, and (FIG. 10H) CRY2. Data were analyzed using one-way or two-way ANOVA with the Newman Keuls post-hoc test or Dunnett's multiple comparison test. (n=6), **p<0.01 and ***p<0.001 vs AD as indicated by a line above the bar. Therefore, it was determined that DPPA4 expression profiles in saliva of FASD and control children to study its role in circadian rhythmicity.

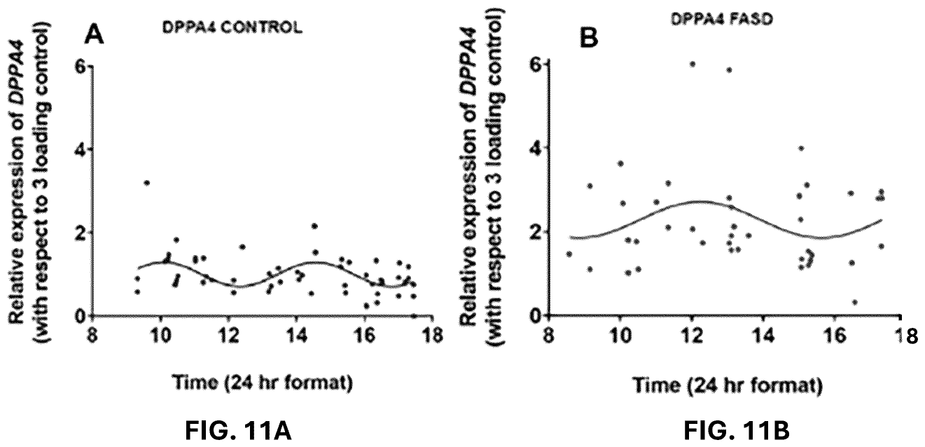

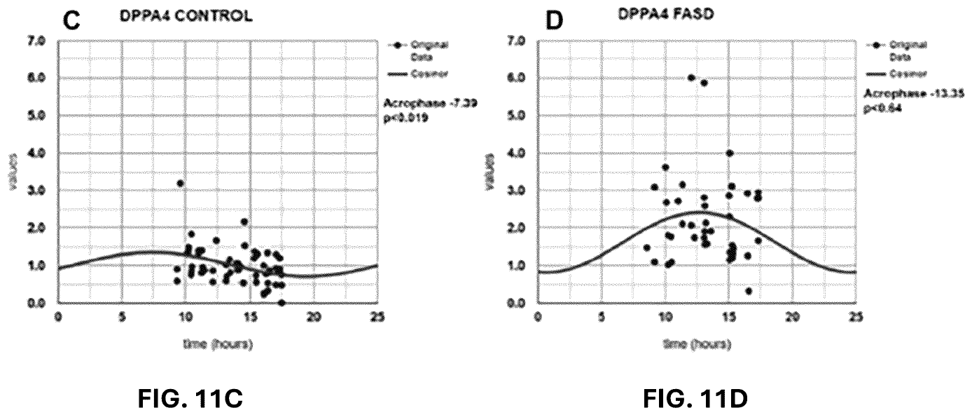

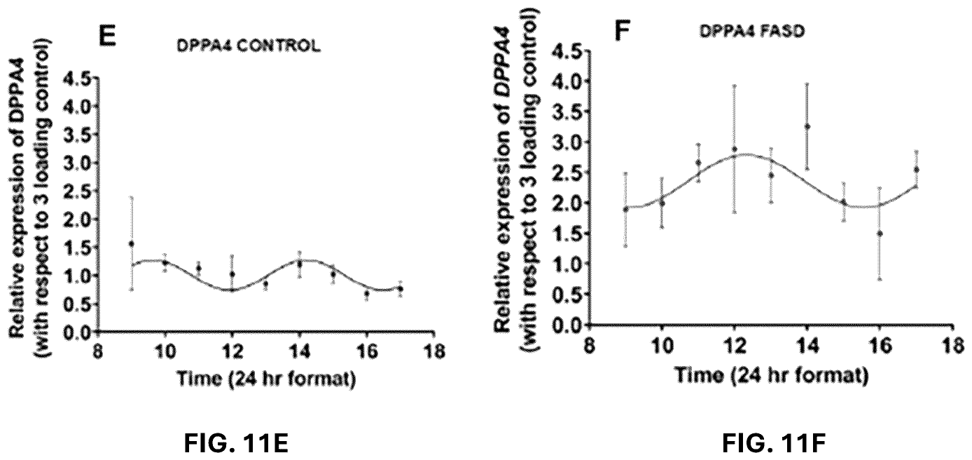

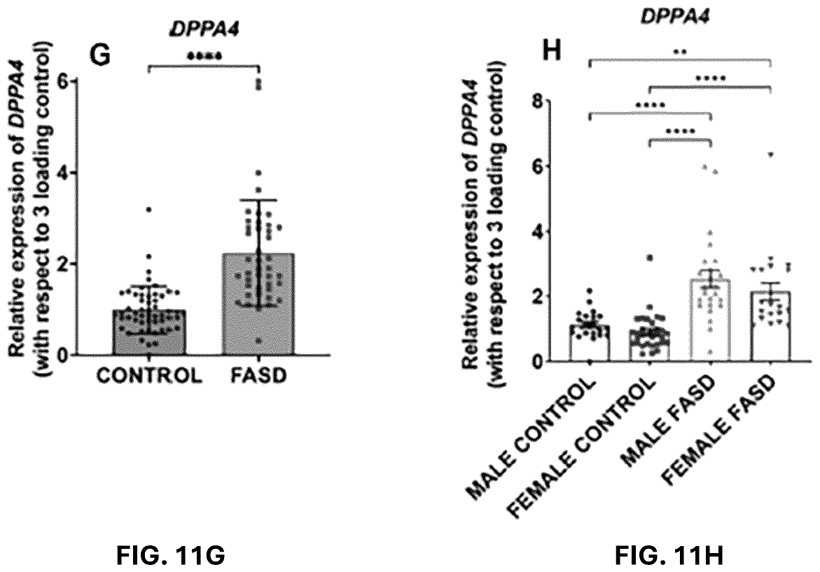

FIGS. 11A-11H shows diagrams that demonstrate that expression levels of DPPA4 in saliva show a circadian rhythm pattern in control but not in FASD. FIG. 11A and FIG. 11B show the effects of AE on circadian expression of DPPA4 levels in saliva for control (FIG. 11A) and FASD (FIG. 11B) groups when plotted individually at various timepoint using a Sinusoidal curve. FIG. 11C and FIG. 11D show the results of a cosinor analysis used to determine the circadian rhythm in control (FIG. 11C) and FASD (FIG. 11D) groups. A zero-amplitude test p-value of <0.05 identified rhythmicity and the Acrophase value shows the time when the rhythm peaked. FIG. 11E-H show gene expression levels of DPPA at various timepoints for control and FASD groups individually as Mean+SEM value at each time point (FIG. 11E-FIG. 11F), combined (FIG. 11G), or separated by sex (FIG. 11H). Data were analyzed using one-way ANOVA; * represents a p-value of <0.05; ** represents a p-value of <0.01; *** represents a p-value of <0.001. These data also suggest that DPPA4 may be a candidate regulator of AE-induced alteration of circadian clock mechanism.

### Example 3

### RNA-Seq Procedure

A 450 μl of saliva sample is used for RNA isolation using the Quick-DNA/RNA Microprep Plus Kit (Zymo Research). The RNA quality is measured with the nanodrop (Thermofisher Scientific). RNA-Seq library preparation and sequencing is carried out from Genewiz of Azenta Life Science. Libraries are then run spread over three lanes of an Illumina HiSeq 2500 using a 150 bp paired end protocol. Sequence reads is trimmed to remove possible adapter sequences and nucleotides with poor quality using Trimmo-matic v. 0.36. The trimmed reads are mapped to the reference genome available on Ensembl using the STAR aligner v. 2.5.2b. Unique gene hit counts are calculated by using feature Counts from the Subread package v. 1.5.2. After extraction of gene hit counts, the gene hit counts table is used for downstream differential expression analysis. Using DESeq2, a comparison of gene expression between groups of samples is performed. The Wald test is used to generate p values and log 2 fold changes. Genes with an adjusted p value of <0.05 and absolute log 2 fold change of >1 are designated as DEGs for each comparison. Raw RNA-seq data are deposited to GEO which are publicly available. Saliva RNA samples were sent for transcriptomic analysis.

### RNA-Seq Data Pre-Processing and Differential Gene Expression Analysis

FastQC (version 0.12) is used for base sequence quality. Paired-end reads are preprocessed, adapters removed using trimmomatic (version 0.40), and trimmed reads are mapped to Ensembl human reference genome (GRCh38.p13) using HISAT2. Low expression transcripts are filtered. Using DESeq2, differentially expressed genes (DEGs) are identified using treatment and group as main effects. Principal component analysis confirms the absence of any batch or any other unintended experimental effects between samples and within groups. Covariate analyses (cell line, subject information, such as sex, age, race) can be included in the regression model for differential expression analysis by DESeq2. DEGs are identified by both FDR<0.05 and fold change of greater than or equal to 1.2 (i.e., log-fold-change of plus or minus 0.26)85-88. MetaseqR (v3.11) determines transcript biotypes79 and brain region enrichment uses Log odds ratios and chi-square tests. Batch effects are accounted for RNA-seq runs.

### Example 4

### Experimental Details—AE Rat Animal Model

The AE animal model is adopted from previously published studies.⁶⁵⁻⁶⁶ Fisher-344 rats from Harlan Laboratories (Indianapolis, IN) and housed them in pairs in Open-type shoe box cages with Bedcob bedding, maintained in a temperature-controlled (22° C.) room under a 12 h light/dark cycle. Animals are bred and on gestation day (GD)7 and were fed ad libitum with a rat chow (AD), liquid diet containing ethanol (AF), or pair-fed (PF) an isocaloric control liquid diet (Bioserve, Frenchtown, NJ). Alcohol-fed animals are first acclimatized to ethanol by feeding them with liquid diets containing increasing concentrations of ethanol (from 1.7 to 5.0% v/v) from GD7 to GD10, and then fed 6.7% v/v ethanol from GD11-GD21. Both AF and PF offspring are cross-fostered on postnatal day 2 (PND2) to untreated lactating dams to avoid any compromised nurturing by their mothers. Further, a litter size of 8 pups/dam was maintained to limit nurturing effect on overall body growth. Pups were weaned on PND21 and housed by sex. A single female offspring is utilized from each experimental group of rat litter. Animal surgery and care were performed in accordance with institutional guidelines and Institutional Animal Care and Use Committee (IACUC) approved protocol (PROTO202100008).

### Example 5

### Experiments Using AE Rat Model-Immunohistochemical Localization of AAV Expression

Rats are anesthetized and transcardially perfused with 4% paraformaldehyde in 1×PBS, brains are post-fix in a 4% paraformaldehyde and then sectioned 30 μm and used for immunohistochemical staining against GFP (AbCam, Cambridge, MA, USA) using standard procedures. Secondary antibodies (conjugated with Alexa 488) were purchased from Molecular Probes (Carlsbad, CA, USA). Animals were excluded from the study if their infection spread was not localized to the SCN or if there was a significant disproportionate amount of infection between both sides.

### Non-invasive Monitoring of Sleep and Circadian Behavior

Rats are individually housed in 18-inch×18-inch polycarbonate cages (Signal Solutions: Sleep Monitoring System for Rats) set with floor sensor pads that are attached to a recording laptop. Activity signals are recorded, identified, and categorized as Wake, non-rapid eye movement sleep (NREMS), or rapid eye movement sleep (REMS) using SleepStats software version 4 (Signal Solutions), binned in 30-minute intervals^{93,94}. All wake, NREMS, and REMS data are presented normalized to each rat's own post-habituation baseline recording, so all measures represent a change from baseline. Diurnal locomotor activity data is further exported as 1 min bins into ClockLab Analysis 6 software (Actimetrics) to generate diurnal activity plots⁹⁴⁻¹⁰¹. Data are presented as mean+/−SEM plotted by Zeitgeber Time (ZT) or light-dark period, and 95% confidence intervals when collapsed by day with α=0.05.

### Example 6

### Experiments Using AE Rat Model—Five-Choice Serial Reaction Time Task (5-CSRTT)

In this study, rats are trained to respond to a brief visual stimulus presented randomly in one of five apertures. The apparatus typically consists of a chamber with a wall containing five holes, each equipped with a light and a food dispenser. The rat initiates a trial by poking its nose into a designated ‘start’ hole. Following a variable delay, a light briefly illuminates one of the five holes. The rat must then respond by poking its nose into the illuminated hole within a specific time frame to receive a food reward. Several parameters can be adjusted, such as the stimulus duration, inter-trial interval, and the punishment for incorrect responses (e.g., a timeout period). Performance metrics include accuracy (correct responses), omission errors (failures to respond), premature responses (responses before the stimulus), and perseverative responses (repeated pokes). The 5-CSRTT is valuable for assessing the effects of pharmacological agents, genetic manipulations, and environmental factors on attentional processes.

### Example 7

### Experiments Using AE Rat Model—Anxiety-Like Behavior

Anxiety-like behavior is measured using the elevated plus maze (EPM) apparatus constructed from black acrylic pieces sitting 60 cm above the floor. The EPM consists of four arms (50 cm L×10 cm W), two arms closed by non-transparent walls (~40 cm high) and two open arms without walls that were joined by a central square (10 cm×10 cm). Behavioral testing is conducted between 1400 and 1700h (during the later portion of the light phase of the light-dark cycle) in a sound attenuated dimly lit room (75 lux open arm; 12 lux closed arm). Rats are acclimated to the behavioral testing room under cover 1 h prior. An individual rat is placed in the center of the EPM and allowed to freely explore for 5 min. Entry is defined as all four paws within an arm. Videos recorded by an overhead high-resolution camera are scored for behaviors including, proportion of open arm entries (number of open entries to total entries×100), proportion of time spent on open arm (time spent on open arm to total time×100), and number of closed entries which are a reliable indicator of locomotor activity.⁶⁷ Other ethologically relevant behaviors are recorded, including rears, head-dips, and stretched attends, and duration of autogrooming.⁶⁸ Anxiety behavior is also measured in the open field (OF) arena, which is constructed from non-transparent black acrylic open-topped box (90 cm L×90 cm W×40 cm H). The behavioral testing is conducted under modified fluorescent lighting (~25 lux at floor level) between 1400 and 1700h (during the later portion of the light-dark cycle). Rats are transported to the room under cover 1 h prior to testing for acclimation. Videos recorded by an overhead high-resolution camera are scored for behaviors including time spent in the center of the arena, center time (20 cm×20 cm square), line crosses in the center, total line crosses, number of rears and stretch-attends, and time spent grooming.⁶⁷ Additionally, the dark/light box (KinderScientific Smart Cage Rack System) is used to assess anxiety-like behaviors. Rats are placed into the dark side (~100 lux) of the box with a small doorway that allowed free exploration of both dark and light sides of the box for 10 minutes. Latency to enter and time spent on the light side of the apparatus are measured.

### Example 8

### Experiments Using AE Rat Model-Stress-Response

Adult male and female AD, PF & AF (treated with AAV-DPPA4-CRISPR/Cas9-KO or AAV-CRISPR/Cas9-V) are subjected to a 60 min restraint stress in a plastic rodent restraint tube and tail blood collected at basal levels before restraint then 15, 30, and 60 min during restraint. After 60 min, the animals are returned to their home cage and tail blood are collected at 30, 60, and 120 min post restraint. Corticosterone and ACTH levels in plasma are quantified using rodent ELISA assays (IBL).

### Example 9

### Experiments Using AE Rat Model-Gene and Protein Expression Measurements

After termination of the experimental paradigm, rats are rapidly sacrificed at 4 hour intervals (ZT0, ZT4, ZT8, ZT12, ZT16, ZT20, ZT24) and their brain tissue are assayed to determine levels of mRNA (RNA-seq+RT-PCR) and protein (ChIP-seq+ChIP assay) in the hypothalamus, hippocampus and central amygdala tissues.

### RNA Isolation, Sequencing and Analysis

RNA are isolated using RNeasy kit from QIAGEN. RNA quantity is assessed using the Nanodrop ND-100 (Thermo Scientific). RNA-Seq library preparation and sequencing are carried out from Genewiz of Azenta Life Science. Using DESeq2, a comparison of gene expression between groups of samples are performed. The Wald test is used to generate p-values and log 2 fold changes. Genes with an adjusted p-value of <0.05 and an absolute log 2 fold change >1 are designated as DEGs for each comparison. Raw RNA-seq data are deposited to GEO. RNA-seq results are verified by the data using RT-PCR assay.

### Example 10

### Experiments Using AE Rat Model-Chromatin Immunoprecipitation (ChIP) Assay/Seq

ChIP assays are conducted using a ChIP Assay Kit (Sigma-Aldrich, Cleveland, OH) following the manufacturer's instructions and the tissue/cell proteins as per the protocol followed previously.⁶⁹ For ChIP-seq, input DNA from the tissue samples and the service from Azenta Life Sciences are used. The library is prepared using TruSeq ChIP kit (Illumina) as the methods used previously.⁷⁰ DNA library are then clustered on to a flowcell, which are loaded on to Illumina HiSeq instrument as per instructions from the manufacturer. The reads are then mapped to the Rattus norvegicus Rnor6.0 reference genome available on Ensembl using the STAR aligner v. 2.5.2b. The reads that can be mapped at a unique genomic location are analyzed further. Enriched regions (peaks) are identified using the MACS software (version 1.4) with an FDR threshold of 5%, and a minimal fold enrichment (ChIP versus input) of 2. Peaks are annotated as promoter if it is found inside the 2 kb region upstream the transcription start site (TSS), or as Gene Body when it is observed inside the transcript [(between the TSS and the Transcription termination site (TTS)]. To assess the statistical significance of the overlap between gene sets, the P-value has been estimated. All statistics are performed using the R statistical software. The peaks that show overlap between the replicates are considered (i.e. that are overlapping). The peak in the vicinity of a gene (50 kb) is also considered. Peaks are then segregated in two categories depending on whether the genes are up or downregulated following modulation in DPPA4. A list of differentially regulated genes (fold change ≥2) are generated and matched with list of differentially expressed genes from RNA seq data.

### Example 11

### Experiments Using AE Rat Model—Data Analysis

Group sizes are based on power analysis with α=0.05, power=0.8, and expected difference in means and SD based on prior experience with these procedures. Specific statistical tests are indicated in each experiment. Experimenters are blind to treatment groups whenever possible. Significance set to p<0.05. Relative mRNA expression levels of the specific genes are measured and calculated by the methods described in the preliminary studies. Data are analyzed with a 2-way ANOVA with group and sex as factors, followed by Bonferroni post hoc tests. Significance level will be set at p<0.05. Cosinor analysis are employed to identify rhythmicity and acrophase value for the rhythm peak timepoint. Sidak post-hoc corrections are used for multiple comparisons when interaction effects were significant. Results are presented as mean+/−SEM for data plotted by Zeitgeber Time (ZT) or light-dark period (CT), and 95% confidence intervals when collapsed by day with significance set at α=0.05. INGENUITY® Pathway Analysis (Qiagen) are used to predict upstream regulators of rhythmic transcripts. Rank-rank hypergeometric overlap (RRHO)83,84 was used to assess the overlap of rhythmic transcripts between unaffected comparison subjects and subjects with FASD.

### Example 12

### Experimental Details

Human Subjects and Saliva Sample Collection: Children aged 6-10 years, with and without heavy prenatal alcohol exposure (PAE), were recruited from the FASD Research Registry, the San Diego State University Center for Behavioral Teratology and the University of California San Diego MotherToBaby research study. Subjects were enrolled as part of a larger study examining sleep and neurobehavioral functioning in individuals with FASD. Two groups of children are recruited and assessed: children with histories of heavy prenatal alcohol exposure (PAE) and typically developing children (CONT). Children of all sexes, races, and ethnicities between 6:0-10:11 years of age were selected based on the inclusion and exclusion criteria³⁶.

Parents collected saliva samples (Zymo Research saliva/sputum collection kit; R1210) at 2 time points at various times of the day for each child. All containers contained DNA/RNA shield solution which protected the DNA/RNA present in the samples. The samples and shield solutions were mixed at a ratio of 1:1 prior to use. A total 53 control and 44 FASD samples were submitted for gene analysis. The overall study was approved by the institutional review boards at University School of Medicine; University of California San Diego/San Diego State University; Rady Children's Hospital. All participants provided informed consent/assent.

Detection of human specific transcripts in saliva: Zymo Research DNA/RNA shield solution (R1210) was used, which protected the DNA/RNA present in the samples. The RNA isolation was done according to the protocol of Zymo Research kit (D7005). The amount of total human RNA present in each sample was determined, followed by normalization based on the amount of human RNA53. The following strategy was taken according to the previous protocol. For analyzing the expression of pan-bacterial rRNA (16S), and human rRNA (18S), total salivary RNA was converted into complementary DNA (cDNA) via reverse transcription using the High-capacity cDNA reverse transcription kit (Applied Biosystem, Life Technologies). The RNA concentration was measured in NanoDrop and total 500 ng of RNA was used as input for all the samples. After reverse transcription, cDNA was diluted and used for qRT-PCR for the detection of 16S and 18S rRNA. The adjustment of input was done according to the previous protocol³⁷. As Nanodrop spectrophotometer only provides total RNA values, which consists of the mixture of human and bacterial RNA, the ratio of the detected raw Ct-values of human rRNA (18S) to pan-bacterial rRNA (16S) was calculated. The rRNA is the representative for all human and bacterial RNA specimens. The ratio was used to calculate fold change value using the following formula: Fold change=2\hat{ }(Ct18S rRNA-Ct16S rRNA). Then, the total amount of RNA loaded for each sample (500 ng) was divided with fold change value which gave the amount of human RNA in individual sample. Finally, normalization was done based on the amount of human RNA (5 ng) and samples are used to synthesized cDNA using oligo d (T) primers. To perform gene expression analysis of human origin only, the eukaryotic messenger RNA (mRNA) was reverse transcribed to cDNA by using the SuperScriptR III First-Strand Synthesis System with Oligo (dT) primers. As a result, pan-bacterial RNA and other human RNA species like tRNA, rRNA were excluded from cDNA conversion. The reverse transcription was performed according to the manufacturer's instructions (RevTsc, T. SuperScript III Reverse Transcriptase (200 U/ML) #18080-085. Manual 1-4 (2004)). The c-DNA was prepared from the RNA of each sample and 1:3 dilution was done using nuclease free water. The realtime qPCR was carried out in 384-well plates. Each well of 384-well plates contained a reaction mixture of 10 μl consisting of 1:3 dilution of c-DNA, primers in Power SYBR Green PCR Master Mix and comparative CT (ΔΔCT) standard run method was carried out in Quant Studio 7 real time PCR system. The expression of genes is expressed in terms of relative quantification using the formula 2\hat{ }−(ΔΔCT)55.

Statistics and reproducibility: The time of collection was not adjusted to Zeitgeber Time (ZT; ZT0 is the equivalent of sunrise). This is because salivary samples were collected throughout the year and the time of sunrise changes during the summer and the winter. All data are presented as CT time. Graph Pad Prism (version 9) software was used for statistical analysis of data. Data are presented as mean±SEM. Cosinor analysis was used to determine acrophase and rhythmicity⁴⁰. The significance between FASD and controls was assessed using unpaired t-test/one-way ANOVA. Newman-Keuls post-hoc analysis were used p<0.05. Additional details regarding quantitation and statistical analysis are provided in the figure legends.

### Results

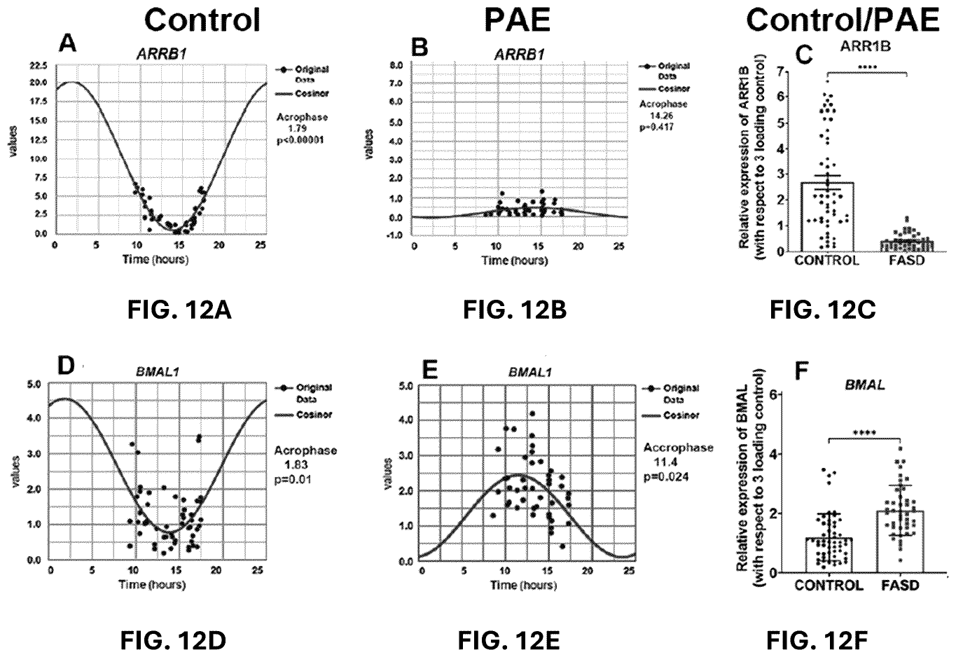

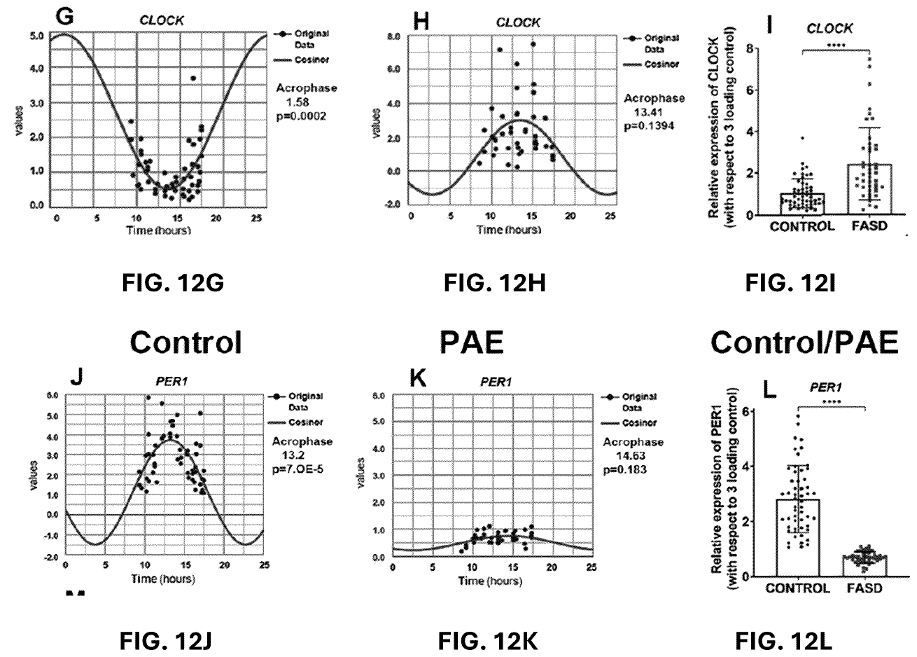

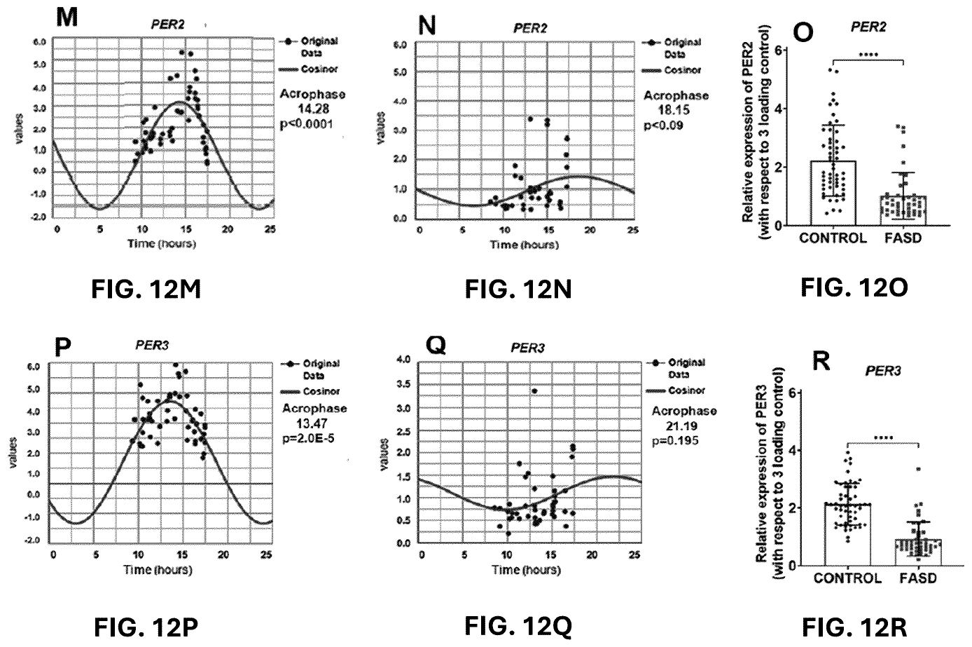

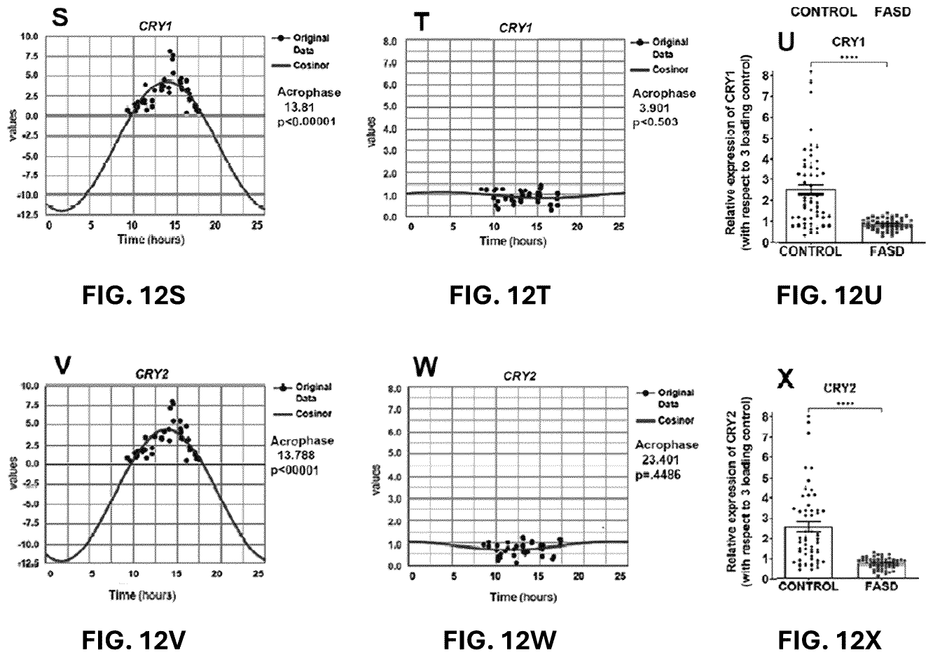

PAE alters core clock genes expression rhythms in saliva of children: First, the expression pattern of a salivary circadian biomarker ARRB126,57 was determined. ARRB1 levels in saliva of control children fluctuated throughout the day in a circadian fashion (Zero-amplitude test p value 0.0001) and showed a peak value at night (acrophase value 1.79h; FIG. 12A), as shown previously in saliva of adult human subjects26.

However, ARRB1 levels in saliva of PAE children fluctuated without a clear circadian pattern (p=0.417) and showed an acrophase value at 14.26 h (FIG. 12B). The overall mean level of ARRB1 was lower in FASD group than that in control group (FIG. 12C). When comparing diurnal changes in the expression levels of core clock genes (BMAL1, CLOCK, PER1,2,3, CRY1,2), the study revealed that expression patterns of primary circadian activator genes BMAL1 (FIGS. 12D, 12E) and CLOCK fluctuated throughout the day (FIGS. 12G, 12H) and showed significant circadian rhythm (FIG. 12G) in control subjects, but not in PAE subjects. Also, the mean levels of BMAL1 and CLOCK genes were higher in PAE group than those in control group (FIG. 12F, 12I). In the case of primary repressor genes of circadian clock, it was determined that all PER (FIG. 12J, M, P) and CRY genes (FIG. 12S, 12T) showed circadian oscillation with peak values in the afternoon in control subjects.

However, in PAE subjects, the circadian oscillations of PER (FIG. 12K, 12N, 12Q) and CRY genes (FIG. 12T, 12W) were disrupted, and the timing of the peak expression levels were shifted to evening/early morning. The overall mean levels of PER1-3 (FIG. 12L, 120, 12R) and CLOCK1,2 genes (FIG. 12U, 12X) were lower in AE group than those in control group. These data suggest that PAE affects the daily circadian oscillation which is maintained by a coordinated transcription-translation feedback loop between the core clock activators (BMAL and CLOCK) and core clock repressors (PER and CRY).

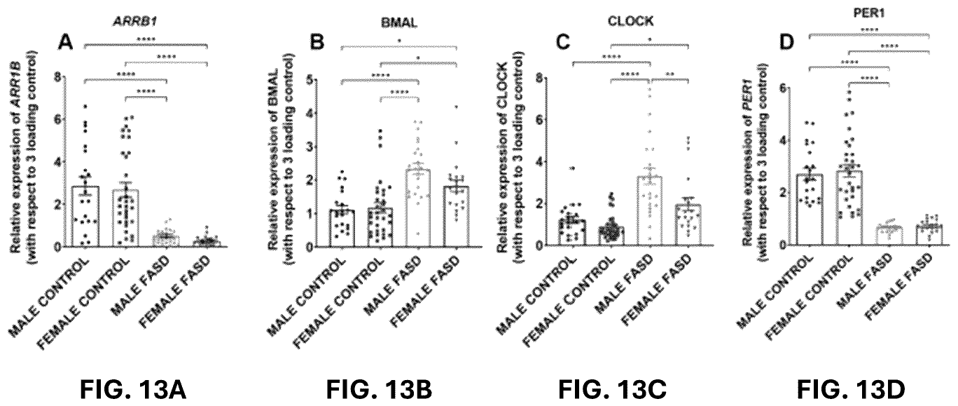

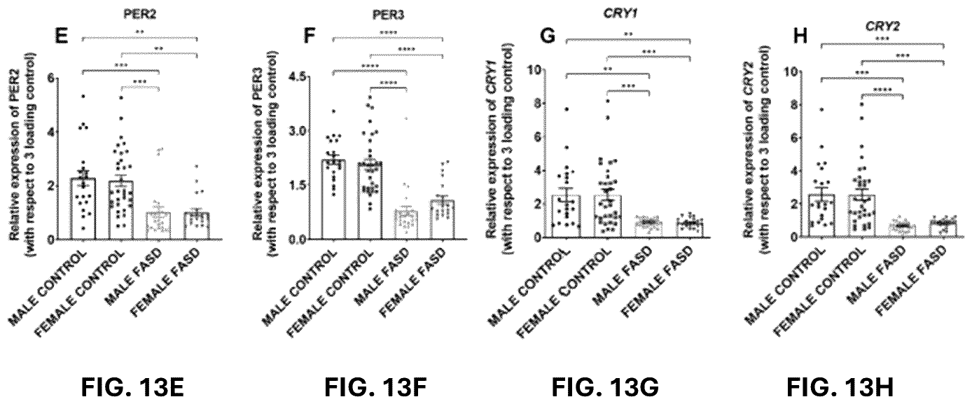

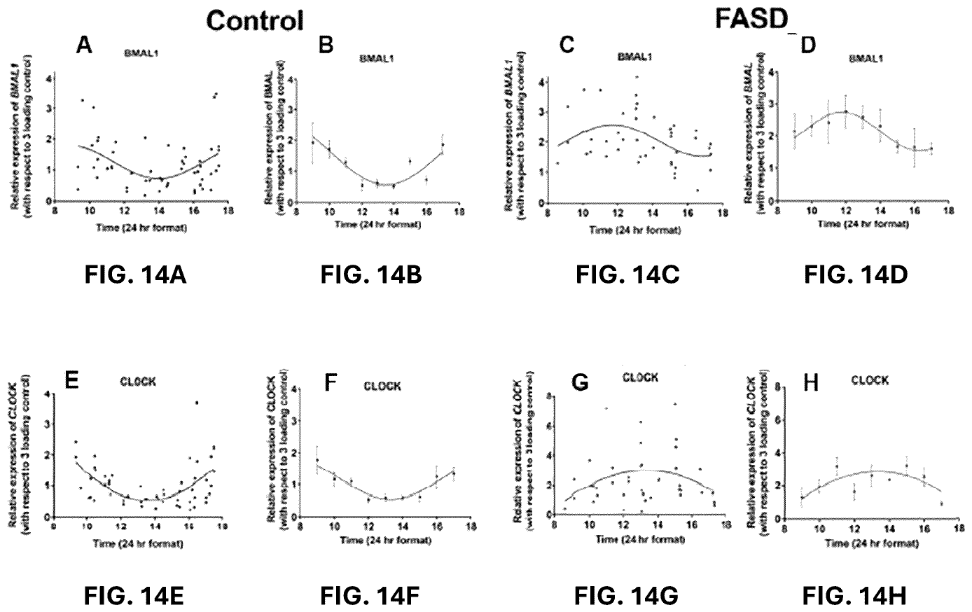

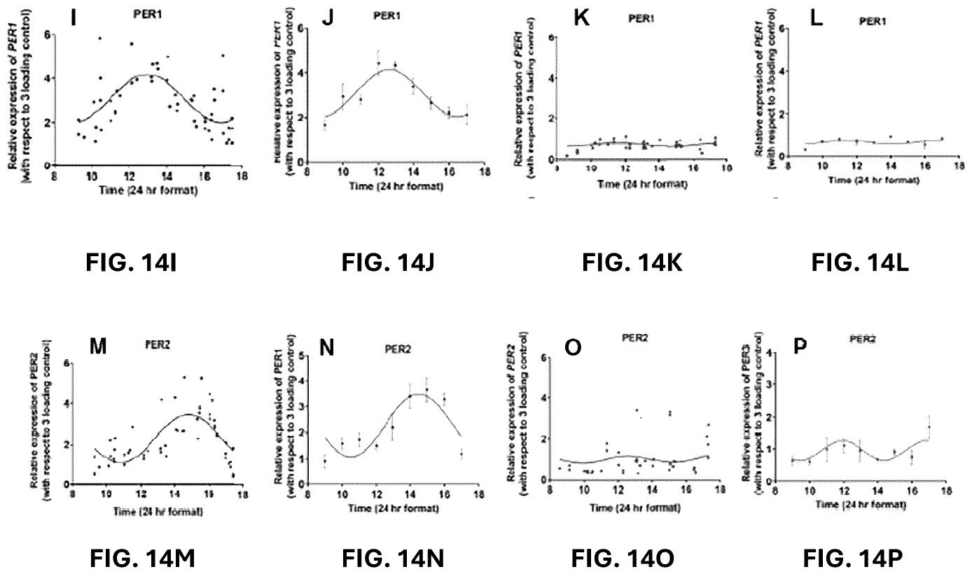

With the exception of CLOCK gene, there were no sex differences in gene expression (FIGS. 13A-13H). In the Cosinor analysis, the individual data points were plotted to get the more accurate Acrophase values. However, to know the variations among different samples collected at a particular time point (e.g., samples collected in between 9 am and 10 am were plotted against 9 am in sign wave graph) (FIGS. 14A-14P). Importantly, the circadian pattern of clock genes in saliva of control subjects maintained a pattern similar to that reported in postmortem adult human brain tissues and in rodent SCN tissues^{20,27}.

### REFERENCES

1. American Academy of Pediatrics. Committee on substance abuse and committee on children with disabilities. Fetal alcohol syndrome and alcohol-related neurodevelopmental disorders. Pediatrics, 106, 358-361 (2000).
2. May, P. A., et al. Prevalence of fetal alcohol Spectrum disorders in 4 US communities. JAMA, 319, 474-482 (2018)
3. O'Connor M J, Paley B. Psychiatric conditions associated with prenatal alcohol exposure. Dev Disabil Res Rev. 2009; 15(3):225-234.
4. Mattson, S. N., Crocker, N., & Nguyen T. T. Fetal alcohol spectrum disorders: neuropsychological and behavioral features. Neuropsychol Rev. 21, 81-101 (2011).
5. Inkelis, S. M., Soja, J., Mattson, S. N., Chambers, C. D., Bhattacharjee, R., & Thomas J D. Characteristics of sleep in children with heavy prenatal alcohol exposure. Alcohol Clin Exp Res (Hoboken). 48,928-943 (2024).
6. Ipsiroglu, O. S., McKellin, W. H., Carey, N., & Loock, C. “They silently live in terror . . . ” why sleep problems and night-time related quality-of-life are missed in children with a fetal alcohol spectrum disorder. Soc Sci Med. 79:76-83 (2013).
7. Mullington, J. M., Haack, M., Toth, M., Serrador, J. M., & Meier-Ewert, H. K. Cardiovascular, inflammatory, and metabolic consequences of sleep deprivation. Prog Cardiovasc Dis. 51:294-302 (2009).
8. Paley B, O'Connor M J. Behavioral interventions for children and adolescents with fetal alcohol spectrum disorders. Alcohol Res Health. 2011; 34(1):64-75.
9. Peadon E, Elliott E J. Distinguishing between attention-deficit hyperactivity and fetal alcohol spectrum disorders in children: clinical guidelines. Neuropsychiatr Dis Treat. 2010; 6:509-515.
10. Jan, J. E., Owens, J. A., Weiss, M. D., Johnson, K. P., Wasdell, M. B., Freeman, R. D., et al. Sleep hygiene for children with neurodevelopmental disabilities. Pediatrics. 122:1343-1350 (2008).
11. Reppert, S. M. & Weaver, D. R. Coordination of circadian timing in mammals. Nature 418:935-941 (2002).
12. Zee, P. C. & Vitiello, M. V. Circadian rhythm sleep disorder: irregular sleep wake rhythm type. Sleep. Med. Clin. 4:213-218 (2009).
13. Tomita, T., Mori, T.& Onishi, Y. β-Arrestin 1 (ARRB1) serves as a molecular marker of the peripheral circadian rhythm. Int J Oral Sci. 2019; 11:32 (2019).
14. Preitner, N. et al. The orphan nuclear receptor REV-ERBalpha controls circadian transcription within the positive limb of the mammalian circadian oscillator. Cell. 110:251-260 (2002).
15. Ueda, H. R. et al. A transcription factor response element for gene expression during circadian night. Nature. 418:534-539 (2002).
16. Honma, S., et al. Dec1 and Dec2 are regulators of the mammalian molecular clock. Nature. 419:841-844 (2002).
17. Yoshitane, H. et al. Functional D-box sequences reset the circadian clock and drive mRNA rhythms. Commun Biol. 2:300 (2019)
18. Pacheco-Bernal, I., Becerril-Perez. F, & Aguilar-Arnal, L. Circadian rhythms in the three-dimensional genome: implications of chromatin interactions for cyclic transcription. Clin Epigenet. 11:79 (2019).
19. Peng, U., Bai, G. & Pang, Y. Roles of NPAS2 in circadian rhythm and disease. Acta Biochim Biophys Sin (Shanghai). 53:1257-1265 (2021).
20. Chen, C. P., Kuhn, P., Advis, J. P. & Sarkar, D. K. Chronic ethanol consumption impairs the circadian rhythm of pro-opiomelanocortin and period genes mRNA expression in the hypothalamus of the male rat. J Neurochem. 88:1547-1554 (2004).
21. Spanagel, R. et al. The clock gene Per2 influences the glutamatergic system and modulates alcohol consumption. Nat Med. 11:35-42 (2005).
22. Spanagel, R., Rosenwasser, A. M., Schumann, G.& Sarkar, D. K. Alcohol consumption and the body's biological clock. Alcohol Clin Exp Res. 29:1550-1557 (2005).
23. Rosenwasser, A. M., Fecteau, M. E. & Logan, R. W. Effects of ethanol intake and ethanol withdrawal on free-running circadian activity rhythms in rats. Physiol Behav. 84:537-542 (2005).
24. Huang, M. C., Ho, C. W., Chen, C. H., Liu, S. C., Chen, C. C., & Leu, S. J. Reduced expression of circadian clock genes in male alcoholic patients. Alcohol Clin Exp Res. 34:1899-904 (2010).
25. Sarkar, D. K. et al. Persistent Changes in Stress-Regulatory Genes in Pregnant Women or Children Exposed Prenatally to Alcohol. Alcohol Clin Exp Res. 43:1887-1897 (2019).
26. Chen, C. P., Kuhn, P., Advis, J. P. & Sarkar, D. K. Prenatal ethanol exposure alters the expression of period genes governing the circadian function of beta-endorphin neurons in the hypothalamus. J Neurochem. 97:1026-1033 (2006).
27. Earnest, D. J., Chen, W. J. & West J. R. Developmental alcohol and circadian clock function. Alcohol Res Health. 25:136-140 (2001).
28. Agapito, M. A., Barreira, J. C., Logan, R. W. & Sarkar, D. K. Evidence for possible period 2 gene mediation of the effects of alcohol exposure during the postnatal period on genes associated with maintaining metabolic signaling in the mouse hypothalamus. Alcohol Clin Exp Res. 37:263-269 (2013).
29. Nakashita, M., et al. Influence of alcohol intake on circadian blood pressure variation in Japanese men: the Ohasama study. Am. J. Hypertens. 22:1171-1176 (2009).
30. Mclachlan, K. et al. Dysregulation of the cortisol diurnal rhythm following prenatal alcohol exposure and early life adversity. Alcohol. 53:9-18. (2016)
31. Boivin, D. B. et al. Circadian clock genes oscillate in human peripheral blood mononuclear cells. Blood. 102:4143-4145 (2003).
32. James, F. O., Boivin, D. B., Charbonneau, S., Belanger, V. & Cermakian, N. Expression of clock genes in human peripheral blood mononuclear cells throughout the sleep/wake and circadian cycles. Chronobiol Int. 24:1009-1034 (2007).
33. Kusanagi, H. et al. Expression profiles of 10 circadian clock genes in human peripheral blood mononuclear cells. Neurosci Res. 61:136-142 (2008).
34. Segal, A. & Wong D. T. Salivary diagnostics: enhancing disease detection and making medicine better. Eur. J. Dent. Educ. Off. J. Assoc. Dent. Educ. Eur. 2008; 12:22-29 (2008).
35. Keiver, K., Bertram, C. P., Orr, A. P. & Clarren, S. Salivary cortisol levels are elevated in the afternoon and at bedtime in children with prenatal alcohol exposure. Alcohol 49:79-87 (2015).
36. Nguyen, T. T., Levy, S. S., Riley, E. P., Thomas, J. D. & Simmons, R. W. Children with heavy prenatal alcohol exposure experience reduced control of isotonic force. Alcohol Clin Exp Res. 37:315-324 (2013).
37. Ostheim P, Tichý A, Sirak I, Davidkova M, Stastna M M, Kultova G, et al. Overcoming challenges in human saliva gene expression measurements. Scientific reports. 10:11147 (2020).
38. Rousseau, B., Murugan, S., Palagani, A. & Sarkar, D. K. Beta 2 adrenergic receptor and mu opioid receptor interact to potentiate the aggressiveness of human breast cancer cell by activating the glycogen synthase kinase 3 signaling. Breast Cancer Res. 24:33 (2022).
39. Doyle, M. M., Murphy, T. E., Miner, B., Pisani, M. A., Lusczek, E. R. & Knauert, M. P. Enhancing cosinor analysis of circadian phase markers using the gamma distribution. Sleep medicine. 92:1-3 (2022).
40. Halberg, F., Tong, Y. L. & Johnson, E. A. Circadian system phase—an aspect of temporalmorphology; procedures and illustrative examples. The Cellular Aspects of Biorhythms. 20e48 (1967).
41. Hayano, J., Jiang, W., Waugh, R., O'Connor, C. & Frid, D, Blumenthal J A. Stability over time of circadian rhythm of variability of heart rate in patients with stable coronary artery disease. Am Heart J. 134:411-8 (1997).
42. Bjarnason. G. A. et al. Circadian expression of clock genes in human oral mucosa and skin: association with specific cell-cycle phases. Am J Pathol. 158:1793-1801 (2001).
43. Papagerakis, S. et al. The circadian clock in oral health and diseases. J Dent Res. 93:27-35 (2014).
44. Liyanage, V. R., Curtis, K., Zachariah, R. M., Chudley, A. E. & Rastegar, M. Overview of the genetic basis and epigenetic mechanisms that contribute to FASD pathobiology. Curr Top Med Chem. 17:808-828 (2017).
45. Basavarajappa, B. S. Epigenetics in fetal alcohol spectrum disorders. Prog Mol Biol Transl Sci. 197:211-239 (2023).
46. Katada, S. & Sassone-Corsi, P. The histone methyltransferase MLL1 permits the oscillation of circadian gene expression. Nat Struct Mol Biol 17:1414-1421 (2010).
47. Papazyan, R., Zhang, Y. & Lazar, M. A. Genetic and epigenomic mechanisms of mammalian circadian transcription. Nat. Struct. Mol. Biol. 23:1045-1052 (2016).
48. Duong, H. A. & Weitz, C. J. Temporal orchestration of repressive chromatin modifiers by circadian clock Period complexes. Nat Struct Mol Biol. 21:126-132 (2014).
49. Li, J. Z. et al., Circadian patterns of gene expression in the human brain and disruption in major depressive disorder. Proc Natl Acad Sci USA. 110:9950-9955 (2013).
50. Takumi, T. et al. A light-independent oscillatory gene mPer3 in mouse SCN and OVLT. EMBO J 17:4753-4759 (1998).
51. Harbour, V. L., Weigl, Y., Robinson, B. & Amir, S. Phase differences in expression of circadian clock genes in the central nucleus of the amygdala, dentate gyrus, and suprachiasmatic nucleus in the rat. PLOS One. 9: e103309 (2014).
52. Kume, K. et al. mCRY1 and mCRY2 are essential components of the negative limb of the circadian clock feedback loop. Cell. 98:193-205 (1999).
53. Zylka, M. J., Shearman, L. P., Weaver, D. R. & Reppert, S. M. Three period homologs in mammals: differential light responses in the suprachiasmatic circadian clock and oscillating transcripts outside of brain. Neuron. 20:1103-1110 (1998).
54. Nakhid, D. et al. Limbic brain subregions associated with mental health symptoms in youth with and without prenatal alcohol exposure. Alcohol Clin Exp Res. 47:2033-2044 (2023).
55. Turner, K. et al. Guided Internet-Based Parent Training for Challenging Behavior in Children With Fetal Alcohol Spectrum Disorder (Strongest Families FASD): Study Protocol for a Randomized Controlled Trial. JMIR Res Protoc. 4: e112 (2015).
56. Alvik, A., Aalen, O. O. & Lindemann, R. Early fetal binge alcohol exposure predicts high behavioral symptom scores in 5.5-year-old children. Alcohol Clin Exp Res. 37:1954-62 (2013).
57. Mclachlan, K. et al. Dysregulation of the cortisol diurnal rhythm following prenatal alcohol exposure and early life adversity. Alcohol. 53:9-18 (2016).
58. Crouse, J. J. et al. Circadian rhythm sleep-wake disturbances and depression in young people: implications for prevention and early intervention. Lancet Psychiatry. 8:813-823 (2021).
59. Robillard, R. et al. Ambulatory sleep-wake patterns and variability in young people with emerging mental disorders. J Psychiatry Neurosci. 40:28-37 (2015).
60. Wengel T, Hanlon-Dearman A C, Fjeldsted B. Sleep and sensory characteristics in young children with fetal alcohol spectrum disorder. J Dev Behav Pediatr. 2011; 32(5):384-392.
61. Chaudhary S, Das U, Sarkar D K. An oncogenic role of developmental pluripotency associated factor 4 in alcohol induced aggressive pituitary tumors. Alcohol Clin Exp Res. 2022; 46:S.454
62. Das U, Chaudhary S, Gangisetty O, Sarkar D K. Prenatal alcohol exposure increases developmental pluripotency associated factor 4 expression via epigenetic modifications; possible involvement in aggressive tumor development. Alcohol Clin Exp Res. 2023; 47:S.459.
63. Chaudhary S, Das U, Jabbar S, Gangisetty O, Rousseau B, Hanft S, Sarkar D K. DPPA4 increases aggressiveness of pituitary neuroendocrine tumors by enhancing cell stemness. Neuro-Oncology (in Revision).
64. Eckersley-Maslin M, Alda-Catalinas C, Blotenburg M, Kreibich E, Krueger C, Reik W. Dppa2 and Dppa4 directly regulate the Dux-driven zygotic transcriptional program. Genes Dev. 2019; 33(3-4):194-208.
65. Jabbar S, Reuhl K, Sarkar D K. Prenatal alcohol exposure increases the susceptibility to develop aggressive prolactinomas in the pituitary gland. Sci Rep. 2018; 8(1):7720.
66. Logan R W, Wynne O, Maglakelidze G, et al. β-Endorphin neuronal transplantation into the hypothalamus alters anxiety-like behaviors in prenatal alcohol-exposed rats and alcohol-non-preferring and alcohol-preferring rats. Alcohol Clin Exp Res. 2015; 39(1):146-157.
67. Logan R W, Seggio J A, Robinson S L, Richard G R, Rosenwasser A M. Circadian wheel-running activity during withdrawal from chronic intermittent ethanol exposure in mice. Alcohol. 2010; 44(3):239-244.
68. Rosenwasser A M, Fecteau M E, Logan R W, Reed J D, Cotter S J, Seggio J A. Circadian activity rhythms in selectively bred ethanol-preferring and nonpreferring rats. Alcohol. 2005; 36(2):69-81.
69. Gangisetty O, Bekdash R, Maglakelidze G, Sarkar D K. Fetal alcohol exposure alters proopiomelanocortin gene expression and hypothalamic-pituitary-adrenal axis function via increasing MeCP2 expression in the hypothalamus. PLOS One. 2014; 9(11):e113228.
70. Deplus R, Delatte B, Schwinn M K, et al. TET2 and TET3 regulate GlcNAcylation and H3K4 methylation through OGT and SET1/COMPASS. EMBO J. 2013; 32(5):645-655.

What is claimed:

1. A method for detection of fetal alcohol spectrum disorders in a subject, comprising:
combining at least one saliva sample from the subject with a DNA/RNA shield solution to form a mixture;
centrifuging the mixture;
adding ethanol to the mixture and further centrifuging the mixture;
washing the mixture with a DNA/RNA buffer solution;
mixing a DNase/RNase-free water solution into the mixture;
collecting RNA from the mixture;
converting the collected RNA into cDNA via reverse transcription;
performing a quantitative reverse transcription polymerase chain reaction (qRT-PCR) on the cDNA to measure gene expression patterns of one or more genes, wherein the one or more genes are selected from the group consisting of BMAL1, CLOCK, ARRB1, PER1, PER2, PER3, CRY1, CRY2, NPAS2, NFL3, NR1D1, DEC1, DEC2, and DBP;
wherein dysregulation of the expression patterns of the one or more genes is indicative of fetal alcohol spectrum disorder in the subject.

2. The method of claim 1, wherein the subject is a human.

3. The method of claim 1, wherein the subject is approximately 6-10 years old.

4. The method of claim 1, wherein the at least one saliva sample and the DNA/RNA shield solution are mixed in approximately a 1:1 ratio.

5. The method of claim 1, wherein the mixture is first centrifuged for approximately 2 minutes.

6. The method of claim 1, wherein the washing of the mixture comprises two washings with the DNA/RNA buffer solution.

7. The method of claim 1, wherein the collected RNA is converted into cDNA via reverse transcription using a high-capacity cDNA reverse transcription kit.

8. The method of claim 1, wherein the one or more genes comprise clock genes.

9. The method of claim 1, wherein the one or more genes include BMAL1 and wherein relative expression of BMAL1 above a normal range is indicative of fetal alcohol spectrum disorder in the subject.

10. The method of claim 1, wherein the one or more genes include CLOCK and wherein relative expression of CLOCK above a normal range is indicative of fetal alcohol spectrum disorder in the subject.

11. The method of claim 1, wherein the one or more genes include at least one of PER1, PER2, CRY1, CRY2, and ARRB1, and wherein relative expression of at least one of PER1, PER2, CRY1, CRY2, and ARRB1 below a normal range is indicative of fetal alcohol spectrum disorder in the subject.

12. The method of claim 1, further comprising:
measuring expression patterns of one or more chromatin modifiers via the qRT-PCR performed on the cDNA, wherein the chromatin modifiers are selected from the group consisting of MLL1, P300, SIRT1, EZH2, HDAC3, and JARID1.

13. A kit for detection of fetal alcohol spectrum disorders in a subject via a saliva sample, the kit comprising:
a saliva collection device;
a DNA/RNA buffer solution;
a DNA/RNA shield solution;
a DNase/RNase-free water solution; and
a cDNA reverse transcription kit configured to convert collected RNA into cDNA.

14. The kit of claim 13, wherein the cDNA reverse transcription kit comprises:
a buffer, a deoxynucleotide triphosphate mix, random primers, and reverse transcriptase.

15. The kit of claim 13, wherein the cDNA reverse transcription kit is a high-capacity cDNA reverse transcription kit.

16. The kit of claim 13, wherein the saliva collection device comprises a collection funnel and a collection tube.
